(19) United States
(12) Patent Application Publication
(10) Pub. No.: US 2012/0167254 A1
(43) Pub. Date: Jun. 28, 2012

(54) Disruption of CKX3 and at least one other CKX gene in a plant or plant cell leads to improved traits

(76) Inventors: Thomas Schmülling, Berlin (DE); Isabel Bartrina Y Manns, Berlin (DE); Tomás Werner, Berlin (DE)

(21) Appl. No.: 13/382,924

(22) PCT Filed: Jul. 9, 2010
(86) PCT No.: PCT/EP10/59880

§ 371 Date: Feb. 11, 2012

(30) Foreign Application Priority Data
Jul. 10, 2009 (EP) 09165164.6

(51) Int. Cl. C12N 15/82; A01H 5/00; C12N 5/10

(52) U.S. Cl. 800/290; 435/419; 800/298; 800/306

(57) ABSTRACT

The present invention is directed to isolated plant cells and transgenic plants comprising a disruption in at least a CKX3 gene and in one further gene encoding for a cytokininoxidase/dehydrogenase and being different from CKX3 well as to methods of producing such transgenic plants and to methods of increasing seed yield in a plant and/or plant height.

In order to be able to supply a continuously growing population with food and other plant-derived products, people have always been interested in improving the productivity in agriculture.

The productivity of a plant can be influenced in various different ways, e.g. by improving plant growth characteristics or by delaying leaf senescence. There are many mechanisms and pathways known which are involved in plant growth and development.

Cytokinin is a plant hormone that plays positive and negative regulatory roles in many aspects of plant growth and development. It stimulates the formation and activity of shoot meristems, is able to establish sink tissues, retard leaf senescence, inhibits root growth and branching, and plays a role in seed germination and stress responses (Mok, D. W. S. & Mok, M. C. (2001) Ann. Rev. Plant Physiol. Mol. Bio. 52, 89-1 18). Analysis of cytokinin-deficient plants has shown that cytokinin plays opposite roles in shoot and root meristems and suggests that the hormone has an essential function in quantitative control of organ growth (Werner T, Motyka V, Laucou V, Smets R, Van Onckelen H, Schmülling T, Plant Cell 2003, 15(11):2532-50; Werner T, Motyka V, Strnad M, Schmülling T, Proc Natl Acad Sci USA 2001, 98(18):10487-92).

Cytokinin oxidases/dehydrogenases (CKX) are an important factor to regulate the homeostasis of the plant hormone cytokinin. The genome of Arabidopsis encodes seven CKX genes, which have distinct expression domains (Werner et al., 2001; Werner et al., 2003). The CKX proteins differ in their subcellular localization and biochemical features (Werner et al., 2003). Overexpression of individual CKX genes established cytokinin-deficient plants and revealed that cytokinin is a positive regulator of the shoot meristem activity and a negative regulator of root meristem activity.

Recently it was shown that in a rice plant inhibition of the function of a particular CKX gene, the rice orthologue to CKX3 of Arabidopsis thaliana, has led to an increase in particle-bearing number of said rice plant (see US 2006/0123507 A1). Although these results are promising, there remains a need for further improving the productivity of plants.

It is an object of the present invention to provide means and methods suitable to produce transgenic plants with improved productivity and/or growth characteristics.

This object is achieved by the present invention as set out in detail below.

The present invention provides isolated plant cells and transgenic plants in which the expression and/or activity of at least two different cytokininoxidase/dehydrogenase genes is inhibited by disruption compared to a control plant cell or a control plant lacking such disruptions, wherein the first cytokininoxidase/dehydrogenase gene is an endogenous gene encoding for CKX3 or an orthologue thereof and the second cytokininoxidase/dehydrogenase gene is an endogenous gene encoding for a cytokininoxidase/dehydrogenase and being different from CKX3 or the orthologue thereof.

Surprisingly it has been found that in a plant simultaneous disruption of the CKX3 gene and a second cytokininoxidase/dehydrogenase gene encoding for one of CKX2, CKX4, CKX5 or CKX6 leads to transgenic plants with a seed yield that is higher than that of a plant lacking such disruptions or transgenic plants where only one cytokininoxidase/dehydrogenase gene is disrupted. Whereas single disruption of CKX3 led to a slight (but not significant) increase in seed yield, as reported in US 2006/0123507 A1, single disruption of CKX5 had no measurable effect on seed yield. Surprisingly the simultaneous disruption of CKX3 and one of CKX2, CKX4, CKX5 or CKX6, but not simultaneous disruption of CKX2 and CKX4 or CKX2 and CKX4 and CKX5 or CKX4 and CKX6 or CKX5 and CKX6, led to a significant increase in seed yield compared to wild type and single disruptions of CKX3 and CKX5. Most significant increase in seed yield was observed for a simultaneous disruption of CKX3 and CKX5. Even more surprisingly it was found that simultaneous disruption of CKX3 and one of CKX2, CKX4, CKX5 or CKX6, in particular of CKX3 and CKX5, led to transgenic plants with significantly improved plant height compared to wild-type plants and transgenic plants comprising single disruptions of CKX3 or CKX5. Thus, simultaneous disruption of at least CKX3 and one further endogenous gene encoding for a cytokininoxidase/dehydrogenase, preferably of CKX1, CKX2, CKX4, CKX5, CKX6 or CKX7 leads to transgenic plants with improved productivity and/or growth characteristics.

In a first aspect the present invention relates to an isolated plant cell comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof;
   and
ii) one further endogenous gene encoding for a cytokininoxidase/dehydrogenase and being different from the gene defined in i);
   wherein said disruptions inhibit expression and/or activity of a product of the at least two disrupted cytokininoxidase/dehydrogenase genes compared to a corresponding control plant cell lacking such disruptions.

In a second aspect, the present invention is directed to a transgenic plant comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof;
   and
ii) one further endogenous gene encoding for a cytokininoxidase/dehydrogenase and being different from the gene defined in i);
   wherein said disruptions inhibit expression and/or activity of a product of the at least two disrupted cytokininoxidase/dehydrogenase genes compared to a corresponding control plant lacking such disruptions. It is understood that for the purpose of the present invention the term “transgenic plant” not only encompasses the plant comprising the disruptions of the invention as such, but also refers to any progeny thereof irrespective of the generation No., i.e. the term “transgenic plant” covers progeny of first generation as well as progeny of the X^{th} generation, provided that said progeny still comprises the disruptions of the invention encompassed by the parent transgenic plant.

In a third aspect, the invention relates to a method of increasing a seed yield in a plant and/or increasing plant height and/or increasing stem thickness relative to a corresponding control plant, the method comprising introducing in a plant a disruption in at least:

i) an endogenous CKX3 gene encoding for a cytokininoxidase/dehydrogenase comprising a polypeptide sequence being identical to or having at least 95% identity with SEQ ID No. 1 or an orthologue thereof;
and
ii) one further endogenous gene encoding for a cytokininoxidase/dehydrogenase and being different from the gene defined in i);
wherein said disruptions inhibit expression and/or activity of a product of the at least two disrupted cytokininoxidase/dehydrogenase genes compared to a corresponding control plant lacking such disruptions.

In a fourth aspect, the present invention is directed to a method for producing a plant with an increased seed yield and/or plant height relative to a corresponding control plant, comprising disrupting in a plant at least:

i) an endogenous CKX3 gene encoding for a cytokininoxidase/dehydrogenase comprising a polypeptide sequence being identical to or having at least 95% identity with SEQ ID No. 1 or an orthologue thereof;
and
ii) one further endogenous gene encoding for a cytokininoxidase/dehydrogenase and being different from the gene defined in i);
wherein said disruptions inhibit expression and/or activity of a product of the at least two disrupted cytokininoxidase/dehydrogenase genes compared to a corresponding control plant lacking such disruptions.

The present invention also relates to an isolated plant cell comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and
ii) in at least one further endogenous gene being:
   a) a CKX1 gene encoding for a cytokininoxidase/dehydrogenase comprising a polypeptide sequence being identical to or having at least 95% identity with SEQ ID No. 13 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 13 over a continuous amino acid sequence of 50 amino acids of SEQ ID No. 13, preferably 100 amino acids of SEQ ID No. 13, more preferably over the whole length of SEQ ID No. 13;
   b) a CKX2 gene encoding for a cytokininoxidase/dehydrogenase comprising a polypeptide sequence being identical to or having at least 95% identity with SEQ ID No. 2 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 2 over a continuous amino acid sequence of 50 amino acids of SEQ ID No. 2, preferably 100 amino acids of SEQ ID No. 2, more preferably over the whole length of SEQ ID No. 2;
   c) a CKX4 gene encoding for a cytokininoxidase/dehydrogenase comprising a polypeptide sequence being identical to or having at least 95% identity with SEQ ID No. 3 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 3 over a continuous amino acid sequence of 50 amino acids of SEQ ID No. 3, preferably 100 amino acids of SEQ ID No. 3, more preferably over the whole length of SEQ ID No. 3;
   d) a CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 4 over a continuous amino acid sequence of 50 amino acids of SEQ ID No. 4, preferably 100 amino acids of SEQ ID No. 4, more preferably over the whole length of SEQ ID No. 4;
   e) a CKX6 gene encoding for a cytokininoxidase/dehydrogenase comprising a polypeptide sequence being identical to or having at least 95% identity with SEQ ID No. 5 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 5 over a continuous amino acid sequence of 50 amino acids of SEQ ID No. 5, preferably 100 amino acids of SEQ ID No. 5, more preferably over the whole length of SEQ ID No. 5;
   or
   f) a CKX7 gene encoding for a cytokininoxidase/dehydrogenase comprising a polypeptide sequence being identical to or having at least 95% identity with SEQ ID No. 6 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 6 over a continuous amino acid sequence of 50 amino acids of SEQ ID No. 6, preferably 100 amino acids of SEQ ID No. 6, more preferably over the whole length of SEQ ID No. 6;
      wherein said disruptions inhibit expression and/or activity of a product of the at least two disrupted cytokininoxidase/dehydrogenase genes compared to a corresponding control plant cell lacking such disruptions.

The present invention also refers to a transgenic plant comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and
ii) in at least one further endogenous gene being:
   a) a CKX1 gene encoding for a cytokininoxidase/dehydrogenase comprising a polypeptide sequence being identical to or having at least 95% identity with SEQ ID No. 13 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 13 over a continuous amino acid sequence of 50 amino acids of SEQ ID No. 13, preferably 100 amino acids of SEQ ID No. 13, more preferably over the whole length of SEQ ID No. 13;
   b) a CKX2 gene encoding for a cytokininoxidase/dehydrogenase comprising a polypeptide sequence being identical to or having at least 95% identity with SEQ ID No. 2 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 2 over a continuous amino acid sequence of 50 amino acids of SEQ ID No. 2, preferably 100 amino acids of SEQ ID No. 2, more preferably over the whole length of SEQ ID No. 2;
   c) a CKX4 gene encoding for a cytokininoxidase/dehydrogenase comprising a polypeptide sequence being identical to or having at least 95% identity with SEQ ID No. 3 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 3 over a continuous amino acid sequence of 50 amino acids of SEQ ID No. 3, preferably 100 amino acids of SEQ ID No. 3, more preferably over the whole length of SEQ ID No. 3;
   d) a CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 4 over a continuous amino acid sequence of 50 amino acids of SEQ ID No. 4, preferably 100 amino acids of SEQ ID No. 4, more preferably over the whole length of SEQ ID No. 4;
   e) a CKX6 gene encoding for a cytokininoxidase/dehydrogenase comprising a polypeptide sequence being identical to or having at least 95% identity with SEQ ID No. 5 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 5 over a continuous amino acid sequence of 50 amino acids of SEQ ID No. 5, preferably 100 amino acids of SEQ ID No. 5, more preferably over the whole length of SEQ ID No. 5;
   or
   f) a CKX7 gene encoding for a cytokininoxidase/dehydrogenase comprising a polypeptide sequence being identical to or having at least 95% identity with SEQ ID No. 6 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 6 over a continuous amino acid sequence of 50 amino acids of SEQ ID No. 6, preferably 100 amino acids of SEQ ID No. 6, more preferably over the whole length of SEQ ID No. 6;
      wherein said disruptions inhibit expression and/or activity of a product of the at least two disrupted cytokininoxidase/dehydrogenase genes compared to a corresponding control plant lacking such disruptions.

The isolated plant cell of the invention and/or the transgenic plant of the invention can comprise a disruption in at least:
i) an endogenous CKX3 gene comprising a nucleic acid sequence being identical to or having at least 95% identity with SEQ ID No. 7 or an orthologue thereof, preferably wherein the orthologue is a gene comprising a nucleic acid sequence with at least 45%, at least 50%, at least 60%, at least 80%, or at least 90% sequence identity to SEQ ID No. 7 over a continuous nucleic acid sequence of 300 nucleotides of SEQ ID No. 7, preferably 500 nucleotides of SEQ ID No. 7, more preferably over the whole length of SEQ ID No. 7;
   and
ii) in at least one further endogenous gene being:
   a) a CKX1 gene comprising a nucleic acid sequence being identical to or having at least 95% identity with SEQ ID No. 14 or an orthologue thereof, preferably wherein the orthologue is an endogenous gene comprising a nucleic acid sequence with at least 45%, at least 50%, at least 60%, at least 80%, or at least 90% sequence identity to SEQ ID No. 14 over a continuous nucleic acid sequence of 300 nucleotides of SEQ ID No. 14, preferably 500 nucleotides of SEQ ID No. 14, more preferably over the whole length of SEQ ID No. 14;
   b) a CKX2 gene comprising a nucleic acid sequence being identical to or having at least 95% identity with SEQ ID No. 8 or an orthologue thereof, preferably wherein the orthologue is an endogenous gene comprising a nucleic acid sequence with at least 45%, at least 50%, at least 60%, at least 80%, or at least 90% sequence identity to SEQ ID No. 8 over a continuous nucleic acid sequence of 300 nucleotides of SEQ ID No. 8, preferably 500 nucleotides of SEQ ID No. 8, more preferably over the whole length of SEQ ID No. 8;
   c) a CKX4 gene comprising a nucleic acid sequence being identical to or having at least 95% identity with SEQ ID No. 9 or an orthologue thereof, preferably wherein the orthologue is an endogenous gene comprising a nucleic acid sequence with at least 45%, at least 50%, at least 60%, at least 80%, or at least 90% sequence identity to SEQ ID No. 9 over a continuous nucleic acid sequence of 300 nucleotides of SEQ ID No. 9, preferably 500 nucleotides of SEQ ID No. 9, more preferably over the whole length of SEQ ID No. 9;
   d) a CKX5 gene comprising a nucleic acid sequence being identical to or having at least 95% identity with SEQ ID No. 10 or an orthologue thereof, preferably wherein the orthologue is an endogenous gene comprising a nucleic acid sequence with at least 45%, at least 50%, at least 60%, at least 80%, or at least 90% sequence identity to SEQ ID No. 10 over a continuous nucleic acid sequence of 300 nucleotides of SEQ ID No. 10, preferably 500 nucleotides of SEQ ID No. 10, more preferably over the whole length of SEQ ID No. 10;
   e) a CKX6 gene comprising a nucleic acid sequence being identical to or having at least 95% identity with SEQ ID No. 11 or an orthologue thereof, preferably wherein the orthologue is an endogenous gene comprising a nucleic acid sequence with at least 45%, at least 50%, at least 60%, at least 80%, or at least 90% sequence identity to SEQ ID No. 11 over a continuous nucleic acid sequence of 300 nucleotides of SEQ ID No. 11, preferably 500 nucleotides of SEQ ID No. 11, more preferably over the whole length of SEQ ID No. 11;
   or
   f) a CKX7 gene comprising a nucleic acid sequence being identical to or having at least 95% identity with SEQ ID No. 12 or an orthologue thereof, preferably wherein the orthologue is an endogenous gene comprising a nucleic acid sequence with at least 45%, at least 50%, at least 60%, at least 80%, or at least 90% sequence identity to SEQ ID No. 12 over a continuous nucleic acid sequence of 300 nucleotides of SEQ ID No. 12, preferably 500 nucleotides of SEQ ID No. 12, more preferably over the whole length of SEQ ID No. 12;
      wherein said disruptions inhibit expression and/or activity of a product of the at least two disrupted cytokininoxidase/dehydrogenase genes compared to a corresponding control plant or control plant cell lacking such disruptions.

Preferably the isolated plant cell of the invention and/or the transgenic plant of the invention comprises a disruption in
i) at least an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and
ii) in an endogenous CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 4 over a continuous amino acid sequence of 50 amino acids of SEQ ID No. 4, preferably 100 amino acids of SEQ ID No. 4, more preferably over the whole length of SEQ ID No. 4.

Preferably the isolated plant cell of the invention and/or the transgenic plant of the invention comprises a disruption in
i) an endogenous CKX3 gene comprising a nucleic acid sequence being identical to or having at least 95% identity with SEQ ID No. 7 or an orthologue thereof, preferably wherein the orthologue is a gene comprising a nucleic acid sequence with at least 45%, at least 50%, at least 60%, at least 80%, or at least 90% sequence identity to SEQ ID No. 7 over a continuous nucleic acid sequence of 300 nucleotides of SEQ ID No. 7, preferably 500 nucleotides of SEQ ID No. 7, more preferably over the whole length of SEQ ID No. 7;
   and
ii) an endogenous CKX5 gene comprising a nucleic acid sequence being identical to or having at least 95% identity with SEQ ID No. 10 or an orthologue thereof, preferably wherein the orthologue is an endogenous gene comprising a nucleic acid sequence with at least 45%, at least 50%, at least 60%, at least 80%, or at least 90% sequence identity to SEQ ID No. 10 over a continuous nucleic acid sequence of 300 nucleotides of SEQ ID No. 10, preferably 500 nucleotides of SEQ ID No. 10, more preferably over the whole length of SEQ ID No. 10.

In the isolated plant cell of the invention and/or in the transgenic plant of the invention, one, more than one or all disruptions of the invention may be facilitated by structural disruption, antisense polynucleotide gene suppression, double stranded RNA induced gene silencing, ribozyme techniques, genomic disruptions, tilling, and/or homologous recombination.

In the isolated plant cell of the invention and/or in the transgenic plant of the invention, one, more than one or all disruptions of the invention may be homozygous disruptions.

The transgenic plant of the invention is preferably selected from the family Brassicaceae, more preferably from the genera Brassica or Arabidopsis.

The present invention is also directed to a cell, organ, tissue or transgenic propagation material derived from a transgenic plant of the invention. Transgenic propagation material encompasses parts of a transgenic plant of the invention such as seeds, tubers, beets/swollen tap roots or fruits derived from a transgenic plant of the invention.

The present invention is also directed to a method of increasing a seed yield of a plant and/or increasing plant height relative to a corresponding control plant, the method comprising introducing in a plant a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and
ii) in at least one further endogenous gene being:
   a) a CKX1 gene encoding for a cytokininoxidase/dehydrogenase comprising a polypeptide sequence being identical to or having at least 95% identity with SEQ ID No. 13 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 13 over a continuous amino acid sequence of 50 amino acids of SEQ ID No. 13, preferably 100 amino acids of SEQ ID No. 13, more preferably over the whole length of SEQ ID No. 13;
   b) a CKX2 gene encoding for a cytokininoxidase/dehydrogenase comprising a polypeptide sequence being identical to or having at least 95% identity with SEQ ID No. 2 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 2 over a continuous amino acid sequence of 50 amino acids of SEQ ID No. 2, preferably 100 amino acids of SEQ ID No. 2, more preferably over the whole length of SEQ ID No. 2;
   c) a CKX4 gene encoding for a cytokininoxidase/dehydrogenase comprising a polypeptide sequence being identical to or having at least 95% identity with SEQ ID No. 3 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 3 over a continuous amino acid sequence of 50 amino acids of SEQ ID No. 3, preferably 100 amino acids of SEQ ID No. 3, more preferably over the whole length of SEQ ID No. 3;
   d) a CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 4 over a continuous amino acid sequence of 50 amino acids of SEQ ID No. 4, preferably 100 amino acids of SEQ ID No. 4, more preferably over the whole length of SEQ ID No. 4;
   e) a CKX6 gene encoding for a cytokininoxidase/dehydrogenase comprising a polypeptide sequence being identical to or having at least 95% identity with SEQ ID No. 5 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 5 over a continuous amino acid sequence of 50 amino acids of SEQ ID No. 5, preferably 100 amino acids of SEQ ID No. 5, more preferably over the whole length of SEQ ID No. 5;
   or
   f) a CKX7 gene encoding for a cytokininoxidase/dehydrogenase comprising a polypeptide sequence being identical to or having at least 95% identity with SEQ ID No. 6 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 6 over a continuous amino acid sequence of 50 amino acids of SEQ ID No. 6, preferably 100 amino acids of SEQ ID No. 6, more preferably over the whole length of SEQ ID No. 6;
      wherein said disruptions inhibit expression and/or activity of a product of the at least two disrupted cytokininoxidase/dehydrogenase genes compared to a corresponding control plant lacking such disruptions.

In a further aspect the present invention is directed to a method for producing a plant, preferably a transgenic plant, with an increased seed yield and/or plant height relative to a corresponding control plant, comprising disrupting in a plant at least:
i) an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and
ii) in at least one further endogenous gene being:
   a) a CKX1 gene encoding for a cytokininoxidase/dehydrogenase comprising a polypeptide sequence being identical to or having at least 95% identity with SEQ ID No. 13 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 13 over a continuous amino acid sequence of 50 amino acids of SEQ ID No. 13, preferably 100 amino acids of SEQ ID No. 13, more preferably over the whole length of SEQ ID No. 13;
   b) a CKX2 gene encoding for a cytokininoxidase/dehydrogenase comprising a polypeptide sequence being identical to or having at least 95% identity with SEQ ID No. 2 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 2 over a continuous amino acid sequence of 50 amino acids of SEQ ID No. 2, preferably 100 amino acids of SEQ ID No. 2, more preferably over the whole length of SEQ ID No. 2;
   c) a CKX4 gene encoding for a cytokininoxidase/dehydrogenase comprising a polypeptide sequence being identical to or having at least 95% identity with SEQ ID No. 3 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 3 over a continuous amino acid sequence of 50 amino acids of SEQ ID No. 3, preferably 100 amino acids of SEQ ID No. 3, more preferably over the whole length of SEQ ID No. 3;
   d) a CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 4 over a continuous amino acid sequence of 50 amino acids of SEQ ID No. 4, preferably 100 amino acids of SEQ ID No. 4, more preferably over the whole length of SEQ ID No. 4;
   e) a CKX6 gene encoding for a cytokininoxidase/dehydrogenase comprising a polypeptide sequence being identical to or having at least 95% identity with SEQ ID No. 5 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 5 over a continuous amino acid sequence of 50 amino acids of SEQ ID No. 5, preferably 100 amino acids of SEQ ID No. 5, more preferably over the whole length of SEQ ID No. 5;
   or
   f) a CKX7 gene encoding for a cytokininoxidase/dehydrogenase comprising a polypeptide sequence being identical to or having at least 95% identity with SEQ ID No. 6 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 6 over a continuous amino acid sequence of 50 amino acids of SEQ ID No. 6, preferably 100 amino acids of SEQ ID No. 6, more preferably over the whole length of SEQ ID No. 6;
      wherein said disruptions inhibit expression and/or activity of a product of the at least two disrupted cytokininoxidase/dehydrogenase genes compared to a corresponding control plant lacking such disruptions.

In the methods of the invention, preferably
i) at least an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and
ii) in an endogenous CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 4 over a continuous amino acid sequence of 50 amino acids of SEQ ID No. 4, preferably 100 amino acids of SEQ ID No. 4, more preferably over the whole length of SEQ ID No. 4,
   can preferably be disrupted.

In the method of the invention, preferably:
i) an endogenous CKX3 gene comprising a nucleic acid sequence being identical to or having at least 95% identity with SEQ ID No. 7 or an orthologue thereof, preferably wherein the orthologue is a gene comprising a nucleic acid sequence with at least 45%, at least 50%, at least 60%, at least 80%, or at least 90% sequence identity to SEQ ID No. 7 over a continuous nucleic acid sequence of 300 nucleotides of SEQ ID No. 7, preferably 500 nucleotides of SEQ ID No. 7, more preferably over the whole length of SEQ ID No. 7;
   and
ii) in at least one further endogenous gene being:
   a) a CKX1 gene comprising a nucleic acid sequence being identical to or having at least 95% identity with SEQ ID No. 14 or an orthologue thereof, preferably wherein the orthologue is an endogenous gene comprising a nucleic acid sequence with at least 45%, at least 50%, at least 60%, at least 80%, or at least 90% sequence identity to SEQ ID No. 14 over a continuous nucleic acid sequence of 300 nucleotides of SEQ ID No. 14, preferably 500 nucleotides of SEQ ID No. 14, more preferably over the whole length of SEQ ID No. 14;
   b) a CKX2 gene comprising a nucleic acid sequence being identical to or having at least 95% identity with SEQ ID No. 8 or an orthologue thereof, preferably wherein the orthologue is an endogenous gene comprising a nucleic acid sequence with at least 45%, at least 50%, at least 60%, at least 80%, or at least 90% sequence identity to SEQ ID No. 8 over a continuous nucleic acid sequence of 300 nucleotides of SEQ ID No. 8, preferably 500 nucleotides of SEQ ID No. 8, more preferably over the whole length of SEQ ID No. 8;
   c) a CKX4 gene comprising a nucleic acid sequence being identical to or having at least 95% identity with SEQ ID No. 9 or an orthologue thereof, preferably wherein the orthologue is an endogenous gene comprising a nucleic acid sequence with at least 45%, at least 50%, at least 60%, at least 80%, or at least 90% sequence identity to SEQ ID No. 9 over a continuous nucleic acid sequence of 300 nucleotides of SEQ ID No. 9, preferably 500 nucleotides of SEQ ID No. 9, more preferably over the whole length of SEQ ID No. 9;
   d) a CKX5 gene comprising a nucleic acid sequence being identical to or having at least 95% identity with SEQ ID No. 10 or an orthologue thereof, preferably wherein the orthologue is an endogenous gene comprising a nucleic acid sequence with at least 45%, at least 50%, at least 60%, at least 80%, or at least 90% sequence identity to SEQ ID No. 10 over a continuous nucleic acid sequence of 300 nucleotides of SEQ ID No. 10, preferably 500 nucleotides of SEQ ID No. 10, more preferably over the whole length of SEQ ID No. 10;
   e) a CKX6 gene comprising a nucleic acid sequence being identical to or having at least 95% identity with SEQ ID No. 11 or an orthologue thereof, preferably wherein the orthologue is an endogenous gene comprising a nucleic acid sequence with at least 45%, at least 50%, at least 60%, at least 80%, or at least 90% sequence identity to SEQ ID No. 11 over a continuous nucleic acid sequence of 300 nucleotides of SEQ ID No. 11, preferably 500 nucleotides of SEQ ID No. 11, more preferably over the whole length of SEQ ID No. 11;
   or
   f) a CKX7 gene comprising a nucleic acid sequence being identical to or having at least 95% identity with SEQ ID No. 12 or an orthologue thereof, preferably wherein the orthologue is an endogenous gene comprising a nucleic acid sequence with at least 45%, at least 50%, at least 60%, at least 80%, or at least 90% sequence identity to SEQ ID No. 12 over a continuous nucleic acid sequence of 300 nucleotides of SEQ ID No. 12, preferably 500 nucleotides of SEQ ID No. 12, more preferably over the whole length of SEQ ID No. 12;
      are disrupted.

In another preferred method of the invention:
i) an endogenous CKX3 gene comprising a nucleic acid sequence being identical to or having at least 95% identity with SEQ ID No. 7 or an orthologue thereof, preferably wherein the orthologue is a gene comprising a nucleic acid sequence with at least 45%, at least 50%, at least 60%, at least 80%, or at least 90% sequence identity to SEQ ID No. 7 over a continuous nucleic acid sequence of 300 nucleotides of SEQ ID No. 7, preferably 500 nucleotides of SEQ ID No. 7, more preferably over the whole length of SEQ ID No. 7;
   and
ii) an endogenous CKX5 gene comprising a nucleic acid sequence being identical to or having at least 95% identity with SEQ ID No. 10 or an orthologue thereof, preferably wherein the orthologue is an endogenous gene comprising a nucleic acid sequence with at least 45%, at least 50%, at least 60%, at least 80%, or at least 90% sequence identity to SEQ ID No. 10 over a continuous nucleic acid sequence of 300 nucleotides of SEQ ID No. 10, preferably 500 nucleotides of SEQ ID No. 10, more preferably over the whole length of SEQ ID No. 10,
   are disrupted.

In the methods of the invention, preferably one, more than one or all disruptions are homozygous disruptions.

The present invention is also directed to an isolated plant cell or a transgenic plant obtainable or obtained by one of the methods of the invention.

In one embodiment, at least one of the disruptions in the isolated plant cell of the invention or in the transgenic plant of the invention is produced by introducing at least one polynucleotide sequence comprising a nucleic acid sequence which has at least about 70%, at least about 75%, at least about 80%, at least about 85%, at least about 90%, at least about 95%, at least about 99%, about 99.5% or more sequence identity to SEQ ID No. 14 (CKX1), SEQ ID No. 7 (CKX3), SEQ ID No. 8 (CKX2), SEQ ID No. 9 (CKX4), SEQ ID No. 10 (CKX5), SEQ ID No. 11 (CKX6), SEQ ID No. 12 (CKX7) or a subsequence thereof, or a complement thereof, into a plant cell, such that the at least one polynucleotide sequence is linked to a promoter in a sense or antisense orientation. In another embodiment, the disruption is introduced into the plant cell or the transgenic plant of the invention by introducing at least one polynucleotide sequence configured for RNA silencing or interference.

In another embodiment, one, more than one or all disruptions in at least one of the above-mentioned endogenous genes comprise insertion of one or more transposons. In yet another embodiment, one, more than one or all disruptions can comprise one or more point mutations in at least one of the above-mentioned endogenous genes.

One, more than one or all disruptions in at least one of the above-mentioned endogenous genes can be homozygous disruptions. Alternatively, one, more than one or all disruptions in at least one of the above-mentioned endogenous genes can be a heterozygous disruption. In certain embodiments, the disruptions in at least one of the above-mentioned endogenous genes can include homozygous disruptions, heterozygous disruptions or a combination of homozygous disruptions and heterozygous disruptions.

Unless defined otherwise, all technical and scientific terms used herein have the same meaning as commonly understood by one of ordinary skill in the art to which the invention pertains. In describing and claiming the present invention, the following terminology will be used in accordance with the definitions set out below.

As used in this specification and the appended claims, the singular forms “a”, “an” and “the” include singular and plural referents unless the content clearly dictates otherwise. Thus, for example, reference to “a cell” includes one cell and a combination of two or more cells, and the like.

The term “plant” refers generically to any of: whole plants, plant parts or organs (e.g. leaves, stems, roots, etc.), shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat), fruit (the mature ovary), plant tissue (e.g. vascular tissue, ground tissue, and the like), tissue culture callus, and plant cells (e.g. guard cells, egg cells, trichomes and the like), and progeny of same. The term “plant” generally means all those organisms which are capable of photosynthesis. Included as plant within the scope of the invention are all genera and species of the higher and lower plants of the plant kingdom. Mature plants means plants at any developmental stage beyond the seedling. Seedling means a young immature plant in an early developmental stage. Annual, perennial, monocotyledonous and/or dicotyledonous plants are preferred. Preference is given to plants of the following plant family: Brassicaceae, in particular to plants of the genera Brassica and Arabidopsis.

Plant cell, as used herein, further includes, without limitation, cells obtained from or found in a plant or a part thereof: seeds, cultures, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues.

The term “disruption” or “disrupted” as used herein means that a gene can be structurally disrupted so as to comprise at least one mutation or structural alteration such that the disrupted gene is incapable of directing the efficient expression of a full-length fully functional gene product. The term “disruption” or “disrupted” also encompasses that the disrupted gene or one of its products can be functionally inhibited or inactivated such that a gene is either not expressed or is incapable of efficiently expressing a full-length and/or fully functional gene product. Functional inhibition or inactivation can result from a structural disruption and/or interruption of expression at either level of transcription or translation. Functional inhibition or inactivation can also be achieved e.g. by methods such as antisense polynucleotide gene suppression, double stranded RNA induced gene silencing, ribozyme techniques, and the like. The inhibition of expression and/or activity can be the result of, e.g. antisense constructs, sense constructs, RNA silencing constructs, RNA interference, genomic disruptions (e.g. transposons, tilling, homologous recombination, etc.), and/or the like. Disruption by functional inhibition also encompasses an inhibition of a gene or one of its products by interaction with a chemical compound, preferably a chemical compound interacting specifically with said gene or gene product. The inhibition of expression and/or activity can be measured by determining the presence and/or amount of transcript (e.g. by Northern blotting or RT-PCR techniques) and/or by determining the presence and/or amount of full length or truncated polypeptide encoded by said gene (e.g. by ELISA or Western blotting) and/or by determining presence and/or amount of cytokininoxidase/dehydrogenase activity of the product of the disrupted cytokininoxidase/dehydrogenase gene. The term “disruption” or “disrupted” as used herein is to be understood that a disruption also encompasses a disruption which is effective only in a part of a plant, in a particular cell type or tissue like e.g. the reproductive meristem or the shoot apex. A disruption may be achieved by interacting with or affecting within a coding region, within a non-coding region and/or within a regulatory region like e.g. a promoter region of a particular gene.

The term “transgenic” refers to a plant that has incorporated nucleic acid sequences, including but not limited to genes, polynucleotides, DNA, RNA, etc., and/or alterations thereto (e.g. mutations, point mutations or the like), which have been introduced into a plant compared to a non-introduced plant by processes which are not essentially biological processes for the production of plants. Thus, the term “transgenic plant” encompasses not only plants comprising non-endogenous nucleic acids, but explicitly refers also to plants that bear mutations in an endogenous gene, e.g. point mutations, which have been introduced into said transgenic plant compared to a non-introduced plant by processes which are not essentially biological processes for the production of plants.

The term “endogenous” relates to any gene or nucleic acid sequence that is already present in a given cell or organism like e.g. a plant. The term “exogenous” relates to any gene or nucleic acid sequences that is not endogenous.

A “transposable element” (TE) or “transposable genetic element” is a DNA sequence that can move from one location to another in a cell. Movement of a transposable element can occur from episome to episome, from episome to chromosome, from chromosome to chromosome, or from chromosome to episome. Transposable elements are characterized by the presence of inverted repeat sequences at their termini. Mobilization is mediated enzymatically by a “transposase”. Structurally, a transposable element is categorized as a “transposon” (TN) or an “insertion sequence element” (IS element) based on the presence or absence, respectively, of genetic sequences in addition to those necessary for mobilization of the element. A mini-transposon or mini-IS element typically lacks sequences encoding a transposase.

The term “nucleic acid” or “polynucleotide” is generally used in its art-recognized meaning to refer to a ribose nucleic acid (RNA) or deoxyribose nucleic acid (DNA) polymer, or analog thereof, e.g., a nucleotide polymer comprising modifications of the nucleotides, a peptide nucleic acid, or the like. In certain applications, the nucleic acid can be a polymer that includes multiple monomer types, e.g., both RNA and DNA subunits. A nucleic acid can be, e.g., a chromosome or chromosomal segment, a vector (e.g., an expression vector), an expression cassette, a naked DNA or RNA polymer, the product of a polymerase chain reaction (PCR), an oligonucleotide, a probe, etc. A nucleic acid can be, e.g., single-stranded and/or double-stranded. Unless otherwise indicated, a particular nucleic acid sequence of the invention optionally comprises or encodes complementary sequences, in addition to any sequence explicitly indicated.

The term “polynucleotide sequence”, “nucleic acid sequence” or “nucleotide sequence” refers to a contiguous sequence of nucleotides in a single nucleic acid or to a representation, e.g., a character string, thereof. That is, a “polynucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

The term “subsequence” or “fragment” is any portion of an entire sequence.

An “expression cassette” is a nucleic acid construct, e.g. vector, such as a plasmid, a viral vector, etc., capable of producing transcripts and, potentially, polypeptides encoded by a polynucleotide sequence. An expression vector is capable of producing transcripts in an exogenous cell, e.g. a bacterial cell, or a plant cell, in vivo or in vitro, e.g. a cultured plant protoplast. Expression of a product can be either constitutive or inducible depending, e.g. on the promoter selected. Antisense, sense or RNA interference or silencing configurations that are not or cannot be translated are expressly included by this definition. In the context of an expression vector, a promoter is said to be “operably linked” or “functionally linked” to a polynucleotide sequence if it is capable of regulating expression of the associated polynucleotide sequence. The term also applies to alternative exogenous gene constructs, such as expressed or integrated transgenes. Similarly, the term operably or functionally linked applies equally to alternative or additional transcriptional regulatory sequences such as enhancers, associated with a polynucleotide sequence.

A polynucleotide sequence, nucleic acid sequence or gene is said to “encode” a sense or antisense RNA molecule, or RNA silencing or interference molecule or a polypeptide, if the polynucleotide sequence can be transcribed (in spliced or unspliced form) and/or translated into the RNA or polypeptide, or a subsequence thereof. The skilled person is well aware of the degeneracy of the genetic code, allowing for a number of different nucleic acid sequences encoding for the same amino acid sequence or polypeptide and has no difficulties in determining whether a given nucleic acid sequence encodes for a given amino acid sequence or polypeptide.

“Expression of a gene” or “expression of a nucleic acid” means transcription of DNA into RNA (optionally including modification of the RNA, e.g. splicing), translation of RNA into a polypeptide (possibly including subsequent modification of the polypeptide, e.g. posttranslational modification), or both transcription and translation, as indicated by the context.

The term “gene” or “gene sequence” is used broadly to refer to any nucleic acid associated with a biological function. Genes typically include coding sequences and/or the regulatory sequences required for expression of such coding sequences. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include promoters and enhancers, to which regulatory proteins such as transcription factors bind, resulting in transcription of adjacent or nearby sequences.

A “polypeptide” is a polymer comprising two or more amino acid residues (e.g. a peptide or a protein). The polymer can additionally comprise non-amino acid elements such as labels, quenchers, blocking groups, or the like and can optionally comprise modifications such as glycosylation or the like. The amino acid residues of the polypeptide can be natural or non-natural and can be unsubstituted, unmodified, substituted or modified.

As used herein the term “cytokininoxidase/dehydrogenase gene” refers to a gene encoding for a polypeptide with cytokininoxidase/dehydrogenase activity. A cytokininoxidase/dehydrogenase is an enzyme that catalyzes the chemical reaction:

N6-dimethylallyladenine+acceptor+H₂O [symbol] adenine+3-methylbut-2-enal+reduced acceptor

The three substrates of this enzyme are N6-dimethylallyladenine, acceptor, and H₂O, whereas its three products are adenine, 3-methylbut-2-enal, and reduced acceptor. Preferably the term “cytokininoxidase/dehydrogenase activity” encompasses the activity of a given polypeptide to catalyse an oxidoreductase reaction with at least one of the cytokinins as substrate. The skilled person is well aware of means and methods to determine whether a given polypeptide has cytokininoxidase/dehydrogenase activity or not and to determine the level of cytokininoxidase/dehydrogenase activity of a particular polypeptide or probe in absolute values and/or relative to another polypeptide or probe. There is ample guidance in the literature how a given polypeptide can be tested for such an activity, see e.g. EC 1.5.99.12. More preferably the term “cytokinin oxidase/dehydrogenase activity” encompasses the activity of a given polypeptide to catalyse an oxidoreductase reaction with at least one of the cytokinins as substrate, preferably with an activity of not less than 30% of the activity of AtCKX3 (CKX3 with SEQ ID No. 1), preferably of not less than 50% of the activity of AtCKX3.

The term “orthologue” as used herein refers to a gene from a species, preferably different from Arabidopsis thaliana, that shows highest similarity, preferably highest sequence identity, to the specified gene of Arabidopsis thaliana because both genes originated from a common ancestor. Preferably the term “orthologue” denotes an endogenous gene encoding for a cytokininoxidase/dehydrogenase and comprising a sequence (polypeptide or nucleic acid) with at least 80%, at least 85%, at least 90%, at least 95%, or at least 99% sequence identity to a given sequence the respective orthologue refers to, preferably over a particular sequence length. More preferably the term “orthologue” denotes an endogenous gene, which is derived from a species different from Arabidopsis thaliana, encoding for a cytokininoxidase/dehydrogenase and comprising a sequence with at least 80%, at least 85%, at least 90%, at least 95%, or at least 99% sequence identity to a given sequence of Arabidopsis thaliana the respective orthologue refers to, preferably over a particular sequence length.

The term “recombinant” indicates that the material (e.g. a cell, a nucleic acid, or a protein) has been artificially or synthetically (non-naturally) altered by human intervention. The alteration can be performed on the material within, or removed from, its natural environment or state. For example, a “recombinant nucleic acid” is one that is made by recombining nucleic acids, e.g. during cloning, DNA shuffling or other procedures; a “recombinant polypeptide” or “recombinant protein” is a polypeptide or protein which is produced by expression of a recombinant nucleic acid. Examples of recombinant cells include cells containing recombinant nucleic acids and/or recombinant polypeptides.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not autonomously replicating.

In the context of the present invention, the term “isolated” refers to a biological material, such as a nucleic acid or a polypeptide, which is substantially free from components that normally accompany or interact with it in its naturally occurring environment. The isolated material optionally comprises material not found with the material in its natural environment, e.g. a cell. For example, if the material is in its natural environment, such as a cell, the material has been placed at a location in the cell (e.g., genome or genetic element) not native to a material found in that environment. For example, a naturally occurring nucleic acid (e.g. a coding sequence, a promoter, an enhancer, etc.) becomes isolated if it is introduced by non-naturally occurring means to a locus of the genome (e.g. a vector, such as a plasmid or virus vector, or amplicon) not native to that nucleic acid. An isolated plant cell, for example, can be in an environment (e.g. a cell culture system, or purified from cell culture) other than the native environment of wild-type plant cells (e.g. a whole plant).

A “promoter”, as used herein, includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells, such as Agrobacterium or Rhizobium. Examples of promoters under developmental control include promoters that preferentially initiate transcription in certain tissues, such as leaves, roots, or seeds or spatially in regions such as endosperm, embryo, or meristematic regions. Such promoters are referred to as “tissue-preferred” or “tissue-specific”. A temporally regulated promoter drives expression at particular times, such as between 0-25 days after pollination. A “cell-type-preferred” promoter primarily drives expression in certain cell types in one or more organs, for example, vascular cells in roots or leaves. An “inducible” promoter is a promoter that is under environmental control and may be inducible or de-repressible. Examples of environmental conditions that may effect transcription by inducible promoters include anaerobic conditions or the presence of light. Tissue-specific, cell-type-specific, and inducible promoters constitute the class of “non-constitutive” promoters. A “constitutive” promoter is a promoter that is active under most environmental conditions and in all or nearly all tissues, at all or nearly all stages of development.

“Transformation”, as used herein, is the process by which a cell is “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to higher eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA.

For the purpose of the present invention, sequence “identity” is objectively determined by any of a number of methods. The skilled person is well aware of these methods and can choose a suitable method without undue burden. A variety of methods for determining relationships between two or more sequences (e.g. identity, similarity and/or homology) are available and well known in the art. The methods include manual alignment, computer assisted sequence alignment and combinations thereof, for example. A number of algorithms (which are generally computer implemented) for performing sequence alignment are widely available or can be produced by one of skill. These methods include, e.g. the local homology algorithm of Smith and Waterman (1981) Adv. Appl. Math. 2: 482; the homology alignment algorithm of Needleman and Wunsch (1970) J. Mol. Biol. 48: 443; the search for similarity method of Pearson and Lipman (1988) Proc. Natl. Acad. Sci. (USA) 85: 2444; and/or by computerized implementations of these algorithms (e.g. GAP, BESTFIT, FASTA, and TFASTA in the Wisconsin Genetics Software Package Release 7.0, Genetics Computer Group, 575 Science Dr., Madison, Wis.).

For example, software for performing sequence identity (and sequence similarity) analysis using the BLAST algorithm is described in Altschul et al. (1990) J. Mol. Biol. 215: 403-410. This software is publicly available, e.g. through the National Center for Biotechnology Information on the world wide web at ncbi. nlm. nih. gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold. These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and, speed of the alignment. The BLASTN program (for nucleotide sequences) uses as defaults a word length (W) of 11, an expectation (E) of 10, a cut-off of 100, M=5, N=−4, and a comparison of both strands. For amino acid sequences, the BLASTP (BLAST Protein) program uses as defaults a word length (W) of 3, an expectation (E) of 10, and the BLOSUM62 scoring matrix (see, Henikoff & Henikoff (1989) Proc. Natl. Acad. Sci. USA 89: 10915).

Additionally, the BLAST algorithm performs a statistical analysis of the similarity between two sequences (see, e.g. Karlin & Altschul (1993) Proc. Natl. Acad. Sci. USA 90: 5873-5787). One measure of similarity provided by the BLAST algorithm is the smallest sum probability (p (N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001.

Another example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35: 351-360. The method used is similar to the method described by Higgins & Sharp (1989) CABIOS 5: 151-153. The program can align, e.g. up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

An additional example of an algorithm that is suitable for multiple DNA, or amino acid, sequence alignments is the CLUSTALW program (Thompson, J. D. et al. (1994) Nucl. Acids. Res. 22: 4673-4680). CLUSTALW performs multiple pairwise comparisons between groups of sequences and assembles them into a multiple alignment based on homology. Gap open and Gap extension penalties can be, e.g., 10 and 0.05 respectively. For amino acid alignments, the BLOSUM algorithm can be used as a protein weight matrix. See, e.g., Henikoff and Henikoff (1992) Proc. Natl. Acad. Sci. USA 89: 10915-10919.

The isolated plant cell or the transgenic plant of the invention can be produced by conventional means like e.g. transformation. The transformation of plant cells and protoplasts can be carried out in essentially any of the various ways known to those skilled in the art of plant molecular biology, including, but not limited to, the methods described herein. See, in general, Methods in Enzymology, Vol. 153 (Recombinant DNA Part D) Wu and Grossman (eds.) 1987, Academic Press. As used herein, the term “transformation” means alteration of the genotype of a host plant or plant cell by the introduction of a nucleic acid sequence, e.g. a “heterologous”, “exogenous” or “foreign” nucleic acid sequence. The heterologous nucleic acid sequence need not necessarily originate from a different source but it will, at some point, have been external to the cell into which is introduced. In addition to Berger, Ausubel and Sambrook, useful general references for plant cell cloning, culture and regeneration include Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St Louis, Mo.) (Sigma-LSRCCC) and, e.g. the Plant Culture Catalogue and supplement (1997) also from Sigma-Aldrich, Inc (St Louis, Mo.) (Sigma-PCCS). Additional details regarding plant cell culture are found in Croy, (ed.) (1993) Plant Molecular Biology Bios Scientific Publishers, Oxford, U. K.

One, more than one or all of the disruptions in at least one of the above-mentioned endogenous genes can be facilitated by introducing and expressing in a plant cell or a plant a transgenic polynucleotide sequence, e.g. in antisense or sense configurations, or RNA silencing or interference configurations, etc, wherein the transgenic polynucleotide sequence comprises a nucleic acid sequence being or being complementary to:
a) a sequence or subsequence of an endogenous CKX3 gene encoding for a cytokininoxidase/dehydrogenase comprising a polypeptide sequence being identical to or having at least 95% identity with SEQ ID No. 1 or an orthologue thereof;
b) a sequence or subsequence of an endogenous CKX1 gene encoding for a cytokininoxidase/dehydrogenase comprising the polypeptide sequence of SEQ ID No. 13 or an orthologue thereof;
c) a sequence or subsequence of an endogenous CKX2 gene encoding for a cytokininoxidase/dehydrogenase comprising a polypeptide sequence being identical to or having at least 95% identity with SEQ ID No. 2 or an orthologue thereof;
d) a sequence or subsequence of an endogenous CKX4 gene encoding for a cytokininoxidase/dehydrogenase comprising a polypeptide sequence being identical to or having at least 95% identity with SEQ ID No. 3 or an orthologue thereof;
e) a sequence or subsequence of an endogenous CKX5 gene encoding for a cytokininoxidase/dehydrogenase comprising a polypeptide sequence being identical to or having at least 95% identity with SEQ ID No. 4 or an orthologue thereof;
f) a sequence or subsequence of an endogenous CKX6 gene encoding for a cytokininoxidase/dehydrogenase comprising a polypeptide sequence being identical to or having at least 95% identity with SEQ ID No. 5 or an orthologue thereof;
g) a sequence or subsequence of an endogenous CKX7 gene encoding for a cytokininoxidase/dehydrogenase comprising a polypeptide sequence being identical to or having at least 95% identity with SEQ ID No. 6 or an orthologue thereof;
   or
h) a sequence or subsequence of an endogenous gene encoding for a cytokininoxidase/dehydrogenase comprising a polypeptide sequence with at least 45%, at least 50%, at least 60%, at least 80%, or at least 90% sequence identity to one of SEQ ID Nos. 1, 2, 3, 4, 5, 6 or 13 over a continuous amino acid sequence of 50 amino acids, preferably 100 amino acids, more preferably over the whole length;
   and comprise a promoter, thereby inhibiting expression and/or activity of at least the disrupted cytokininoxidase/dehydrogenase gene compared to a corresponding control plant cell or plant lacking such disruptions (e.g. its non-transgenic parent or a non-transgenic plant of the same species). The transgenic polynucleotide sequence can be introduced by techniques including, but not limited to, e.g. electroporation, micro-projectile bombardment, Agrobacterium-mediated transfer, or other available methods. In certain aspects of the invention, the polynucleotide is linked to the promoter in a sense orientation or in an antisense orientation or is configured for RNA silencing or interference.

Relevant literature describing the application of homology-dependent gene silencing includes: Jorgensen, Trends Biotechnol. 8 (12): 340-344 (1990); Flavell, Proc. Natl. Acad. Sci. (USA) 91: 3490-3496 (1994); Finnegan et al., Bio/Technology 12: 883-888 (1994); Neuhuber et al., Mol. Gen. Genet. 244: 230-241 (1994); Flavell et al. (1994) Proc. Natl. Acad. Sci. USA 91: 3490-3496; Jorgensen et al. (1996) Plant Mol. Biol. 31: 957-973; Johansen and Carrington (2001) Plant Physiol. 126: 930-938; Broin et al. (2002) Plant Cell 14: 1417-1432; Stoutjesdijk et al. (2002) Plant Physiol. 129: 1723-1731; Yu et al. (2003) Phytochemistry 63: 753-763; and U.S. Pat. Nos. 5,034,323, 5,283,184, and 5,942,657.

Alternatively, another approach to gene silencing can be with the use of antisense technology (Rothstein et al. in Plant Mol. Cell. Biol. 6: 221-246 (1989); Liu et al. (2002) Plant Physiol. 129: 1732-1743 and U.S. Pat. Nos. 5,759,829 and 5,942, 657. Use of antisense nucleic acids is well known in the art. An antisense nucleic acid has a region of complementarity to a target nucleic acid, e.g. a particular genomic gene sequence, an mRNA, or cDNA. The antisense nucleic acid can be RNA, DNA, a PNA or any other appropriate molecule. A duplex can form between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g. in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al (1991) Symp Soc. Exp Biol 45: 117-127; de Lange et al., (1995) Curr Top Microbiol Immunol 197: 57-75; Hamilton et al. (1995) Curr Top Microbiol Immunol 197: 77-89; Finnegan et al., (1996) Proc Natl Acad Sci USA 93: 8449-8454; Uhlmann and A. Pepan (1990), Chem. Rev. 90: 543; P. D. Cook (1991), Anti-Cancer Drug Design 6: 585; J. Goodchild, Bioconjugate Chem. 1 (1990) 165; and, S. L. Beaucage and R. P. Iyer (1993), Tetrahedron 49: 6123; and F. Eckstein, Ed. (1991), Oligonucleotides and Analogues—A Practical Approach, IRL Press.

Catalytic RNA molecules or ribozymes can also be used to inhibit expression of particular selected genes. It is possible to design ribozymes that specifically pair with virtually any desired target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the constructs. A number of classes of ribozymes have been identified. For example, one class of ribozymes is derived from a number of small circular RNAs that are capable of self-cleavage and replication in plants. The RNAs can replicate either alone (viroid RNAs) or with a helper virus (satellite RNAs). Examples of RNAs include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes has been described. See, e.g., Haseloff et al. (1988) Nature, 334: 585-591.

Another method to inactivate a particular selected gene by inhibiting expression is by sense suppression. Introduction of expression cassettes in which a nucleic acid is configured in the sense orientation with respect to the promoter has been shown to be an effective means by which to block the transcription of a desired target gene. See, e.g., Napoli et al. (1990), The Plant Cell 2: 279-289, and U.S. Pat. Nos. 5,034,323, 5,231,020 and 5,283,184.

Disruptions of the invention can also be produced by using RNA silencing or interference (RNAi), which can also be termed post-transcriptional gene silencing (PTGS) or co-suppression. In the context of this invention, “RNA silencing” (also called RNAi or RNA-mediated interference) refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g. histone acetylation and heterochromatin formation leading to inhibition of transcription of the target mRNA). In “RNA interference” the presence of the single-stranded or double-stranded RNA in the cell leads to endonucleolytic cleavage and then degradation of the target mRNA.

In one embodiment, a transgene (e.g. a sequence and/or subsequence of a gene or coding sequence of interest) is introduced into a plant cell to disrupt one or more genes by RNA silencing or interference (RNAi). For example, a sequence or subsequence (the transgene) includes a small subsequence, e.g. about 21-25 bases in length, a larger subsequence, e.g. about 25-100 or about 100-2000 (or about 200-1500, about 250-1000, etc.) bases in length, and/or the entire coding sequence or gene selected from or being complementary to:
a) a sequence or subsequence of an endogenous CKX3 gene encoding for a cytokininoxidase/dehydrogenase comprising a polypeptide sequence being identical to or having at least 95% identity with SEQ ID No. 1 or an orthologue thereof;
b) a sequence or subsequence of an endogenous CKX1 gene encoding for a cytokininoxidase/dehydrogenase comprising a polypeptide sequence being identical to or having at least 95% identity with SEQ ID No. 13 or an orthologue thereof;
c) a sequence or subsequence of an endogenous CKX2 gene encoding for a cytokininoxidase/dehydrogenase comprising a polypeptide sequence being identical to or having at least 95% identity with SEQ ID No. 2 or an orthologue thereof;
d) a sequence or subsequence of an endogenous CKX4 gene encoding for a cytokininoxidase/dehydrogenase comprising a polypeptide sequence being identical to or having at least 95% identity with SEQ ID No. 3 or an orthologue thereof;
e) a sequence or subsequence of an endogenous CKX5 gene encoding for a cytokininoxidase/dehydrogenase comprising a polypeptide sequence being identical to or having at least 95% identity with SEQ ID No. 4 or an orthologue thereof;
f) a sequence or subsequence of an endogenous CKX6 gene encoding for a cytokininoxidase/dehydrogenase comprising a polypeptide sequence being identical to or having at least 95% identity with SEQ ID No. 5 or an orthologue thereof;
g) a sequence or subsequence of an endogenous CKX7 gene encoding for a cytokininoxidase/dehydrogenase comprising a polypeptide sequence being identical to or having at least 95% identity with SEQ ID No. 6 or an orthologue thereof;
   or
h) a sequence or subsequence of an endogenous gene encoding for a cytokininoxidase/dehydrogenase comprising a polypeptide sequence with at least 45%, at least 50%, at least 60%, at least 80%, or at least 90% sequence identity to one of SEQ ID Nos. 1, 2, 3, 4, 5, 6 or 13 over a continuous amino acid sequence of 50 amino acids, preferably 100 amino acids, more preferably over the whole length.

Preferably, a transgene includes a region in the sequence or subsequence that is about 21-25 bases in length with at least 80%, at least 90%, or at least 99% identity to a subsequence of one of the sequences with the SEQ ID No. 7, 8, 9, 10, 11, 12 or 14.

Use of RNAi for inhibiting gene expression in a number of cell types (including, e.g. plant cells) and organisms, e.g. by expression of a hairpin (stem-loop) RNA or of the two strands of an interfering RNA, for example, is well described in the literature, as are methods for determining appropriate interfering RNA (s) to target a desired gene, and for generating such interfering RNAs. For example, RNA interference is described e.g. in US patent application publications 20020173478, 20020162126, and 20020182223 and in Cogoni and Macino (2000), “Post-transcriptional gene silencing across kingdoms” Genes Dev., 10: 638-643; Guru T. (2000), “A silence that speaks volumes” Nature 404: 804-808; Hammond et al., (2001), “Post-transcriptional Gene Silencing by Double-stranded RNA” Nature Rev. Gen. 2: 110-119; Napoli et al., (1990) “Introduction of a chalcone synthase gene into Petunia results in reversible co-suppression of homologous genes in trayas.” Plant Cell 2: 279-289; etc.

The polynucleotide sequence(s) or subsequence(s) to be expressed to induce RNAi can be expressed, e.g., under control of a constitutive promoter, an inducible promoter, or a tissue specific promoter. Expression from a tissue-specific promoter can be advantageous in certain embodiments.

One, more than one or all disruptions in at least one of the above-mentioned endogenous genes can be introduced by, e.g. transposon-based gene inactivation. For example, the inactivating step comprises producing one or more mutations in a gene being:
i) an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and/or
ii) in at least one further endogenous gene being:
   a) a CKX1 gene encoding for a cytokininoxidase/dehydrogenase comprising a polypeptide sequence being identical to or having at least 95% identity with SEQ ID No. 13 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 13 over a continuous amino acid sequence of 50 amino acids of SEQ ID No. 13, preferably 100 amino acids of SEQ ID No. 13, more preferably over the whole length of SEQ ID No. 13;
   b) a CKX2 gene encoding for a cytokininoxidase/dehydrogenase comprising a polypeptide sequence being identical to or having at least 95% identity with SEQ ID No. 2 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 2 over a continuous amino acid sequence of 50 amino acids of SEQ ID No. 2, preferably 100 amino acids of SEQ ID No. 2, more preferably over the whole length of SEQ ID No. 2;
   c) a CKX4 gene encoding for a cytokininoxidase/dehydrogenase comprising a polypeptide sequence being identical to or having at least 95% identity with SEQ ID No. 3 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 3 over a continuous amino acid sequence of 50 amino acids of SEQ ID No. 3, preferably 100 amino acids of SEQ ID No. 3, more preferably over the whole length of SEQ ID No. 3;
   d) a CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 4 over a continuous amino acid sequence of 50 amino acids of SEQ ID No. 4, preferably 100 amino acids of SEQ ID No. 4, more preferably over the whole length of SEQ ID No. 4;
   e) a CKX6 gene encoding for a cytokininoxidase/dehydrogenase comprising a polypeptide sequence being identical to or having at least 95% identity with SEQ ID No. 5 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 5 over a continuous amino acid sequence of 50 amino acids of SEQ ID No. 5, preferably 100 amino acids of SEQ ID No. 5, more preferably over the whole length of SEQ ID No. 5;
   or
   f) a CKX7 gene encoding for a cytokininoxidase/dehydrogenase comprising a polypeptide sequence being identical to or having at least 95% identity with SEQ ID No. 6 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 6 over a continuous amino acid sequence of 50 amino acids of SEQ ID No. 6, preferably 100 amino acids of SEQ ID No. 6, more preferably over the whole length of SEQ ID No. 6;
      wherein the one or more mutations in the gene sequence comprise one or more transposon insertions and wherein the disruptions inhibit expression and/or activity of at least the disrupted cytokininoxidase/dehydrogenase gene compared to a corresponding control plant cell or plant lacking such disruptions. For example, the one or more mutations comprise a homozygous disruption in one or more genes mentioned above or the one or more mutations comprise a heterozygous disruption in one or more genes mentioned above or a combination of both homozygous disruptions and heterozygous disruptions.

Transposons were first identified in maize by Barbara McClintock in the late 1940s. The Mutator family of transposable elements, e.g. Robertson's Mutator (Mu) transposable elements, are typically used in plant gene mutagenesis, because they are present in high copy number (10-100) and insert preferentially within and around genes.

Transposable elements can be categorized into two broad classes based on their mode of transposition. These are designated Class I and Class II; both have applications as mutagens and as delivery vectors. Class I transposable elements transpose by an RNA intermediate and use reverse transcriptases, i.e. they are retroelements. There are at least three types of Class I transposable elements, e.g. retrotransposons, retroposons, SINE-like elements. Retrotransposons typically contain LTRs, and genes encoding viral coat proteins (gag) and reverse transcriptase, RnaseH, integrase and polymerase (pol) genes. Numerous retrotransposons have been described in plant species. Such retrotransposons mobilize and translocate via a RNA intermediate in a reaction catalyzed by reverse transcriptase and RNase H encoded by the transposon. Examples fall into the Tyl-copia and Ty3-gypsy groups as well as into the SINE-like and LINE-like classifications. A more detailed discussion can be found in Kumar and Bennetzen (1999) Plant Retrotransposons in Annual Review of Genetics 33: 479.

In addition, DNA transposable elements such as Ac, Taml and En/Spm are also found in a wide variety of plant species, and can be utilized in the invention.

Transposons (and IS elements) are common tools for introducing mutations in plant cells. These mobile genetic elements are delivered to cells, e.g. through a sexual cross, transposition is selected for and the resulting insertion mutants are screened, e.g. for a phenotype of interest. The disrupted genes can then be introduced into other plants by crossing the isolated or transgenic plants with a non-disrupted plant, e.g. by a sexual cross. Any of a number of standard breeding techniques can be used, depending upon the species to be crossed. The location of a TN within a genome of an isolated or transgenic plant can be determined by known methods, e.g. sequencing of flanking regions as described herein. For example, a PCR reaction from the plant can be used to amplify the sequence, which can then be diagnostically sequenced to confirm its origin. Optionally, the insertion mutants are screened for a desired phenotype, such as the inhibition of expression or activity of agene of interest compared to a control plant.

TILLING can also be used to identify a disruption of the present invention. TILLING is Targeting Induced Local Lesions In Genomes. See, e.g., McCallum et al., (2000), “Targeting Induced Local Lesions In Genomes (TILLING) for Plant Functional Genomics” Plant Physiology 123: 439-442; McCallum et al., (2000), “Targeted screening for induced mutations” Nature Biotechnology 18: 455-457; and, Colbert et al., (2001), “High-Throughput Screening for Induced Point Mutations” Plant Physiology 126: 480-484.

TILLING combines high density point mutations with rapid sensitive detection of the mutations. Typically, ethyl methanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes. For example, DNA from M2 plants is pooled and mutations in a gene of interest are detected by detection of heteroduplex formation. Typically, DNA is prepared from each M2 plant and pooled. The desired gene is amplified by PCR. The pooled sample is then denatured and annealed to allow formation of heteroduplexes. If a mutation is present in one of the plants; the PCR products will be of two types: wild-type and mutant. Pools that include the heteroduplexes are identified by separating the PCR reaction, e.g. by Denaturing High Performance Liquid Chromatography (DPHPLC). DPHPLC detects mismatches in heteroduplexes created by melting and annealing of heteroallelic DNA. Chromatography is performed while heating the DNA. Heteroduplexes have lower thermal stability and form melting bubbles resulting in faster movement in the chromatography column. When heteroduplexes are present in addition to the expected homoduplexes, a double peak is seen. As a result, the pools that carry the mutation in a gene of interest are identified. Individual DNA from plants that make up the selected pooled population can then be identified and sequenced. Optionally, the plant possessing a desired mutation in a gene of interest can be crossed with other plants to remove background mutations.

Other mutagenic methods can also be employed to introduce a disruption of the invention. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Other detection methods for detecting mutations in a gene of interest can be employed, e.g. capillary electrophoresis (e.g. constant denaturant capillary electrophoresis and single-stranded conformational polymorphism). In another example, heteroduplexes can be detected by using mismatch repair enzymology (e.g. CEL I endonuclease from celery). CEL I recognizes a mismatch and cleaves exactly at the 3′ side of the mismatch. The precise base position of the mismatch can be determined by cutting with the mismatch repair enzyme followed by, e.g. denaturing gel electrophoresis. See, e.g. Oleykowski et al., (1998), “Mutation detection using a novel plant endonuclease” Nucleic Acid Res. 26: 4597-4602; and, Colbert et al., (2001), “High-Throughput Screening for Induced Point Mutations” Plant Physiology 126: 480-484.

The plant containing the desired disruption(s) of the invention can be crossed with other plants to introduce the disruptions into another plant. This can be done using standard breeding techniques.

Homologous recombination can also be used to introduce a disruption of the invention. Homologous recombination has been demonstrated in plants. See, e.g. Puchta et al. (1994), Experientia 50: 277-284; Swoboda et al. (1994), EMBO J. 13: 484-489; Offring a et al. (1993), Proc. Natl. Acad. Sci. USA 90: 7346-7350; Kempin et al. (1997) Nature 389: 802-803; and, Terada et al., (2002), “Efficient gene targeting by homologous recombination in rice” Nature Biotechnology, 20 (10): 1030-1034.

Homologous recombination can be used to induce targeted gene modifications by specifically targeting a gene of interest in vivo. Mutations in selected portions of a selected gene sequence (including 5′ upstream, 3′ downstream, and intragenic regions) such as e.g.:
i) an endogenous CKX3 gene encoding for a cytokininoxidase/dehydrogenase comprising a polypeptide sequence being identical to or having at least 95% identity with SEQ ID No. 1 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to SEQ ID No. 1 over a continuous amino acid sequence of 50 amino acids of SEQ ID No. 1, preferably 100 amino acids of SEQ ID No. 1, more preferably over the whole length of SEQ ID No. 1;
   and/or
ii) in at least one further endogenous gene being:
   a) a CKX1 gene encoding for a cytokininoxidase/dehydrogenase comprising a polypeptide sequence being identical to or having at least 95% identity with SEQ ID No. 13 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 13 over a continuous amino acid sequence of 50 amino acids of SEQ ID No. 13, preferably 100 amino acids of SEQ ID No. 13, more preferably over the whole length of SEQ ID No. 13;
   b) a CKX2 gene encoding for a cytokininoxidase/dehydrogenase comprising a polypeptide sequence being identical to or having at least 95% identity with SEQ ID No. 2 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 2 over a continuous amino acid sequence of 50 amino acids of SEQ ID No. 2, preferably 100 amino acids of SEQ ID No. 2, more preferably over the whole length of SEQ ID No. 2;
   c) a CKX4 gene encoding for a cytokininoxidase/dehydrogenase comprising a polypeptide sequence being identical to or having at least 95% identity with SEQ ID No. 3 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 3 over a continuous amino acid sequence of 50 amino acids of SEQ ID No. 3, preferably 100 amino acids of SEQ ID No. 3, more preferably over the whole length of SEQ ID No. 3;
   d) a CKX5 gene encoding for a cytokininoxidase/dehydrogenase comprising a polypeptide sequence being identical to or having at least 95% identity with SEQ ID No. 4 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 4 over a continuous amino acid sequence of 50 amino acids of SEQ ID No. 4, preferably 100 amino acids of SEQ ID No. 4, more preferably over the whole length of SEQ ID No. 4;
   e) a CKX6 gene encoding for a cytokininoxidase/dehydrogenase comprising a polypeptide sequence being identical to or having at least 95% identity with SEQ ID No. 5 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 5 over a continuous amino acid sequence of 50 amino acids of SEQ ID No. 5, preferably 100 amino acids of SEQ ID No. 5, more preferably over the whole length of SEQ ID No. 5;
   or
   f) a CKX7 gene encoding for a cytokininoxidase/dehydrogenase comprising a polypeptide sequence being identical to or having at least 95% identity with SEQ ID No. 6 or an orthologue thereof, preferably wherein the orthologue is an endogenous gene encoding for a cytokininoxidase/dehydrogenase comprising a polypeptide sequence with at least 45%, at least 50%, at least 60%, at least 80%, or at least 90% sequence identity to one of SEQ ID No. 6 over a continuous amino acid sequence of 50 amino acids of SEQ ID No. 6, preferably 100 amino acids of SEQ ID No. 6, more preferably over the whole length of SEQ ID No. 6;
      are made in vitro and introduced into the desired plant using standard techniques. The mutated gene will interact with the target wild-type gene in such a way that homologous recombination and targeted replacement of the wild-type gene will occur in transgenic plants.

Isolated plant cells and/or transgenic plants of the invention, which can be consumed by humans and animals, may also be used, for example directly or after preparation known per se, as foodstuffs or feedstuffs.

The invention further relates to the use of the above-described isolated plant cells and/or transgenic plants of the invention and of the cells, cell cultures, parts, such as, for example, roots, leaves, etc., in the case of transgenic plant organisms, and transgenic propagation material such as seeds, tubers, beets/swollen tap roots or fruits derived from them for the production of food- or feedstuffs, pharmaceuticals or fine chemicals.

In the following the present invention is further described by way of examples.

### FIGURES

FIG. 1: shows positions of T-DNA and transposon insertions in the ckx mutants. The insertional mutants were identified by PCR screening, and the site of insertion determined by DNA sequencing of the border fragment. Black boxes represent exons, white boxes represent introns, and triangles indicate T-DNA insertions. G, GABI-KAT T-DNA-collection; S, Salk T-DNA-collection; T, Torrey Mesa T-DNA-collection; Z, ZIGIA transposon collection.

FIG. 2 shows characterization of ckx T-DNA and transposon insertion alleles. Absence of CKX gene expression in insertional mutants. RNA from 10-d-old seedlings was used as template for the RT-PCR analysis. Actin2 was amplified as control.

FIG. 3 shows cytokinin content in ckx3 ckx5 mutant and wild-type inflorescences. 0.5 g of Arabidopsis inflorescences per sample was harvested and pooled 30 DAG. Five independent biological samples were harvested for each genotype. Data shown are mean values of cytokinin content [pmol/g fresh weight]±s.d.; n=5. tZ, trans-zeatin; tZR; trans-zeatin riboside; tZRMP, trans-zeatin riboside 5′-monophosphate; tZ9G, trans-zeatin 9-glucoside; tZROG, trans-zeatin riboside O-glucoside; iP, N⁶-(Δ² isopentenyl)adenine; iPR, N⁶-(Δ² isopentenyl)adenosine; iPRMP, N⁶-(Δ² isopentenyl)adenosine 5′-monophosphate; iP9G, N⁶-(Δ² isopentenyl)adenine 9-glucoside.

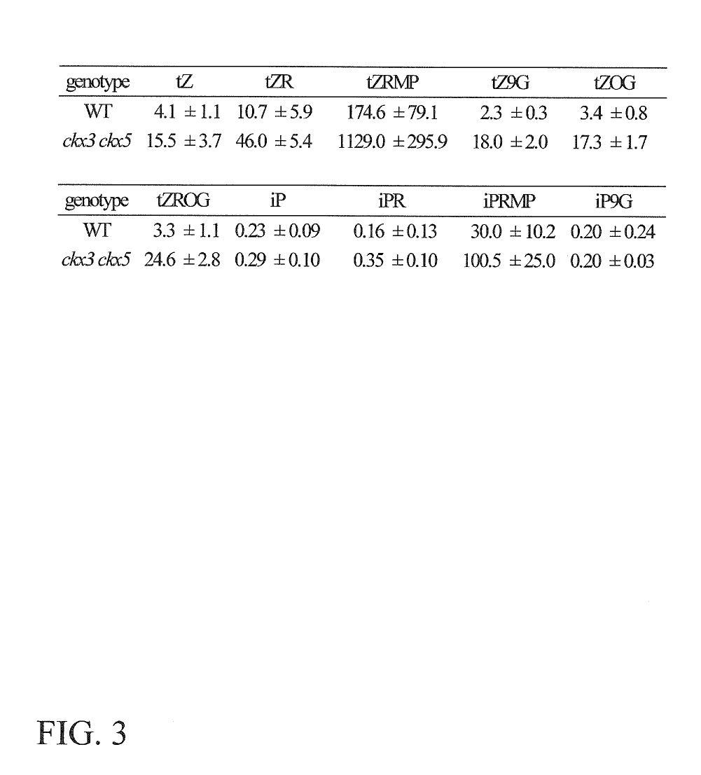

FIG. 4 shows a comparison of shoot morphology from wild-type and ckx mutants. Number of siliques generated by wild type and ckx mutants on the main stem during one life cycle. Wild-type plants had formed 54.7 siliques (100%). Plant height of Arabidopsis wild type and ckx mutants at the end of the flowering time. The height of wild-type plants was 39.5 cm (100%). Data represent mean values±s.d. (n=13-17). *, P<0.01 compared to wild type; =P<0.01 compared to ckx3.

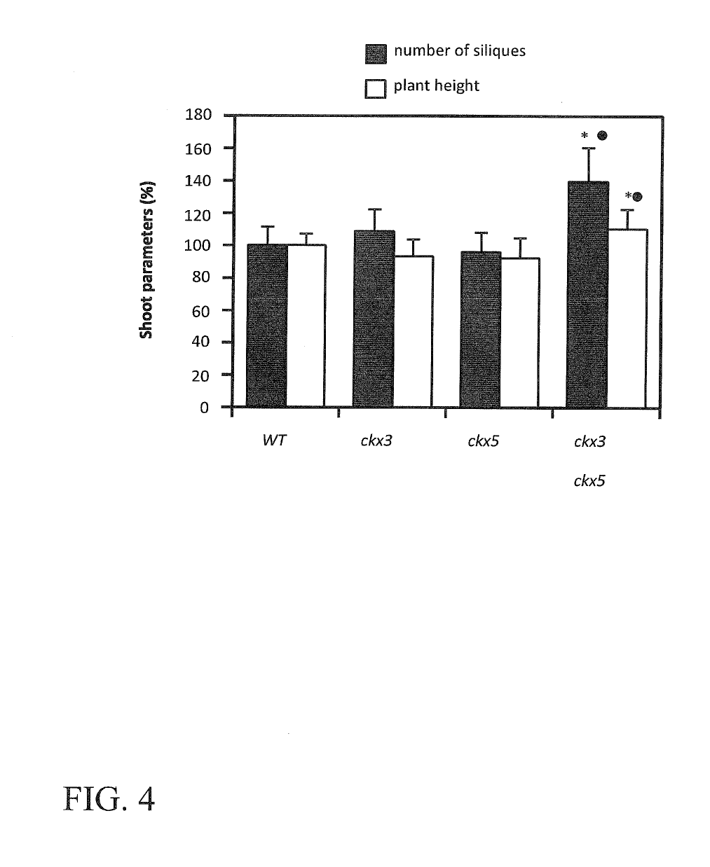

FIG. 5 shows flower phenotype and seed yield of ckx mutants. a, b, Stage 13 flowers (a) and the corresponding gynoecia (b) From left to right are shown wild type, ckx3, ckx5 and ckx3 ckx5. c, Petal surface of ckx mutants, stage 14 flowers, 39 DAG (n=30). d, Number of ovules per gynoeceum (n=12). e, Seed yield of wild type and ckx3 ckx5 under growth chamber conditions (n=30). Data represent mean values±s.d., P<0.01 compared to wild type.

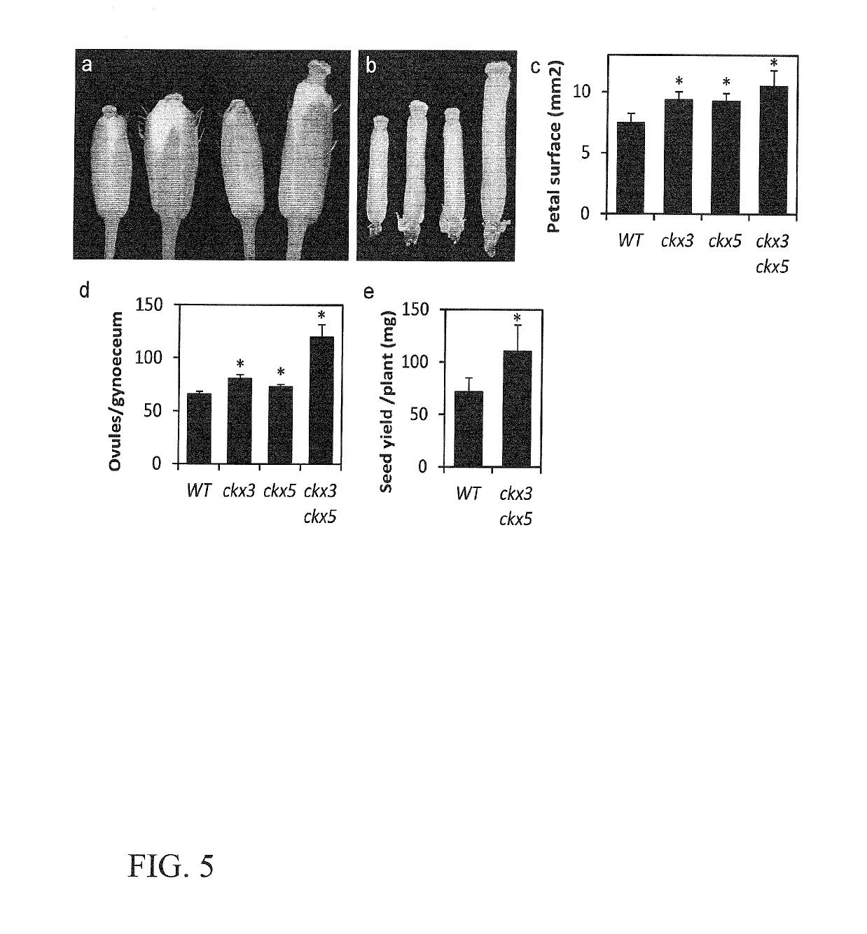

FIG. 6 shows number of siliques generated by wild type and ckx mutants on the main stem during one life cycle (n=15). Wild-type plants had formed 54.7 siliques (100%). Data represent mean values±s.d. *, P<0.01 in comparison to WT. , P<0.01 in comparison to ckx3.

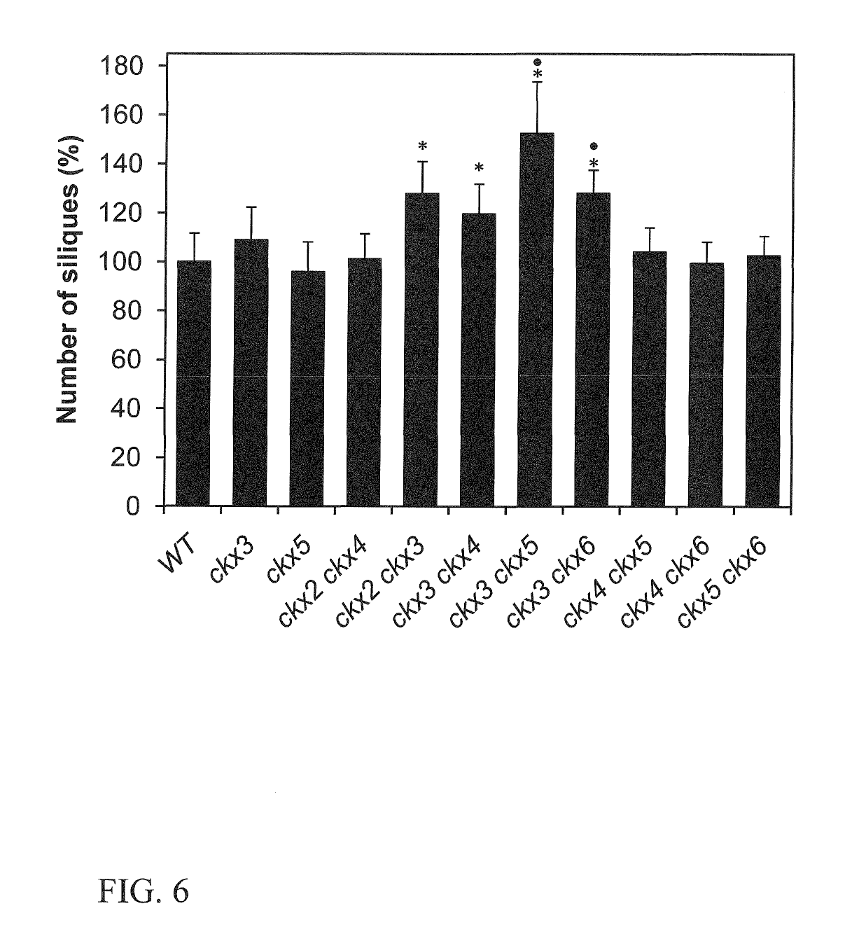

FIG. 7 shows young ovules of wild type and ckx3 ckx5 mutant. Staging of ovules according to Schneitz et al. Scale bar: 10 μm. The number of ovules is increased in ckx3 ckx5 mutants compared to wild type plants.

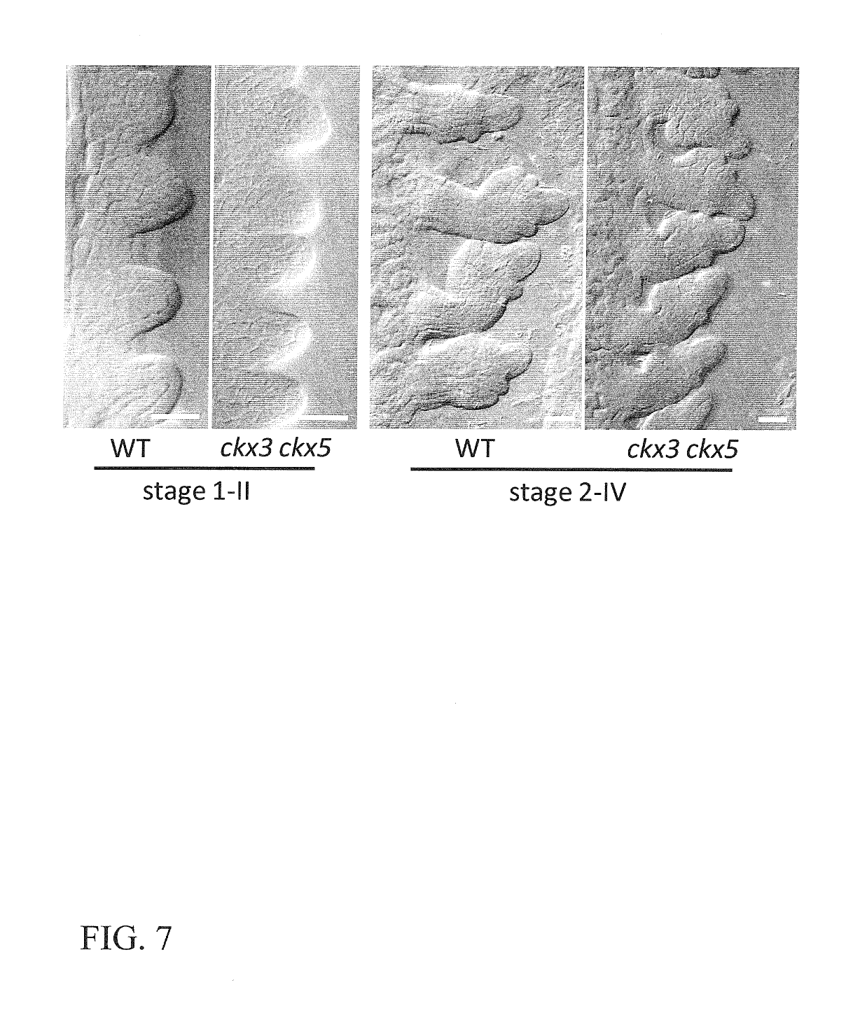

### EXAMPLES

### Methods

### Plant Material and Growth Conditions

The Columbia (Col-0) ecotype of Arabidopsis thaliana was used as the wild type. The T-DNA insertion mutants ckx2-S1 (SALK_{—}068485), ckx3-S1 (SALK_{—}050938), ckx4-S1 (SALK_{—}055204), ckx5-S1 (SALK_{—}064309), and ckx6-S1 (SALK_{—}070071) were from the Salk Institute Genomic Analysis Laboratory (Alonso et al., (2003) Science 301, 653-657), the transposon insertion mutant ckx4-Z was from the ZIGIA transposon collection (Baumann E, Lewald J, Saedler H, Schulz B, Wisman E (1998) Successful PCR-based reverse genetic screens using an En-1-mutagenised Arabidopsis thaliana population generated via single-seed descent. Theoretical and Applied Genetics 97: 729-734), ckx5-G2 (Line ID 332B10) and ckx7-G1 (Line ID 363C02) were from the GABI-KAT collection (Rosso, M. G., Li, Y., Strizhov, N., Reiss, B., Dekker, K., and Weisshaar, B. (2003) Plant Mol. Biol. 53, 247-259) and ckx7-T1 (SAIL_{—}515_A07) was from the Torrey Mesa Research Institute (now Syngenta). To verify the T-DNA insertion genomic primer 1 and left border primer, and to find homozygous lines genomic primer 1 and 2 were used (table 1). Double mutants were obtained by crossing and insertions were confirmed by genomic PCR with gene-specific and T-DNA border primers (Table 1). The mutant line ckx4-Z was not used as a crossing partner. Plants were grown in the greenhouse on soil at 22° C. under long-day conditions (16 h light/8 h dark). For seed yield measurement plants were grown in growth chambers (Percival AR-66L) on soil at 24° C. in ˜100 pE and 65% humidity under long-day conditions.

### Analysis of CKX Expression

Total RNA was extracted from seedlings according to Verwoerd et al. (Verwoerd et al., 1989). The RNA was treated with RNase-free DNase I (Fermentas, St. Leon-Rot, Germany) at 37° C. for 30 min. One microliter of 25 mM EDTA was added at 65° C. for 10 min. RNA (0.5 μg) was used for a RT-PCR reaction. All used primer pairs span the respective T-DNA insertion site (Table 2). In all RT-PCR reactions, the primers for Actin2 were used as controls. RT-PCR was performed with the One-Step RT-PCR kit (Qiagen, Hilden, Germany) according to the manufacturer's instructions. The PCR comprised 35 cycles of 30 s at 94° C., 30 s at 57° C., and 2 min at 72° C.

### Scanning Electron Microscopy

Scanning electron microscopy was performed as described by Krupková et al. (Krupková, E., Immerzeel, P., Pauly, M., and Schmülling, T. (2007) Plant J. 50, 735-750) using a LEO 430 microscope (Zeiss, Oberkochen, Germany).

### Cytokinin Measurement

Plants were grown on soil until the main inflorescence was about 10 cm high (approx. 30 DAG). For each sample ˜0.5 g of inflorescences with stage 1 to stage 15 flowers (Smyth, D. R., Bowman, J. L., and Meyerowitz, E. M. (1990) Plant Cell 2, 755-767) were pooled and five independent samples were collected and analyzed for each genotype. The cytokinin content was determined by ultra-performance liquid chromatography-electrospray tandem mass spectrometry (Novák, O., Hauserová, E., Amakorová, P., Dole\check{z}al, K., and Strnad, M. (2008) Phytochemistry 69, 2214-2224).

### Petal Surface

The area of petals was measured from digital images of dissected organs with the Scion Image program (Scion Corporation, Frederick, Md., USA).

### Determination of Final Plant Height and Yield Parameters

The final plant height and the number of siliques on the main stem were determined after termination of flowering. For analysis of seed yield, plants were put into paper bags after termination of flowering. After plants were kept dry for additional three weeks, total seed weight was determined.

### Light Microscopy

For ovule counting and observation gynoecia were cleared and mounted as described (Malamy and Benfey, 1997). All samples were viewed with an Axioskop 2 plus microscope (Zeiss, Jena, Germany).

### Ovules Counting and Staging

Ovules of wild type and ckx3 ckx5 mutants were prepared, analysed and staged as described in Schneitz er al (1995). Wild-type ovule development in Arabidopsis thaliana: a light microscope study of cleared whole-mount tissue. Plant J. 7, /31-749.

It appeared that the capacity of the placenta tissue to initiate ovule primordia is enhanced in ckx3 ckx5 mutants compared to wild type plants, resulting in a higher overall number and density of ovules and seeds within the carpels.

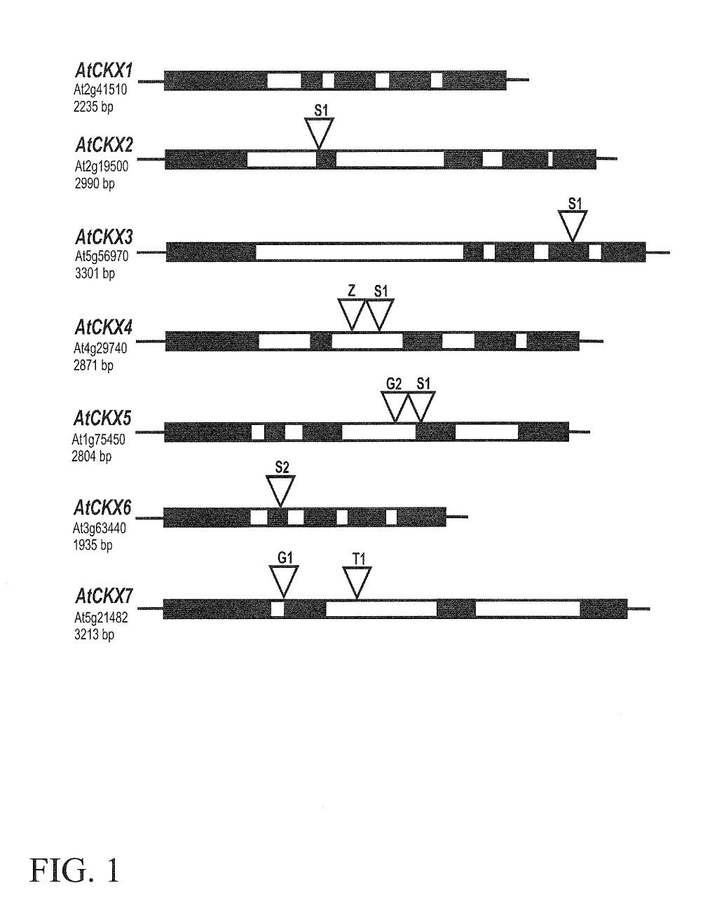

TABLE 1
Primer used to identify T-DNA inserions shown in FIG. 1.

| | | | Left border primer to the |
|---|---|---|---|
| | genomic primer 1 | genomic primer 2 | T-DNA insertion |
| ckx2- | GAATGGTGGAATTGGTGGTC | GCGAGCATGTCAACATTTCA | TGGTTCACGTAGTGGGCCATCG |
| S1 | (SEQ ID No. 15) | (SEQ ID No. 16) | (SEQ ID No. 17) |
| ckx3- | TCAAAAGCCTCCCAATTGTC | CTCGGCTAAAGACGGAGTTG | TGGTTCACGTAGTGGGCCATCG |
| S1 | (SEQ ID No. 18) | (SEQ ID No. 19) | (SEQ ID No. 20) |
| ckx4- | CTCTGCCGCTTCTCACGACTTCGGTA | CATAAACCCTGGAGCGAAACCTAGAG | TGGTTCACGTAGTGGGCCATCG |
| S1 | (SEQ ID No. 21) | (SEQ ID No. 22) | (SEQ ID No. 23) |
| ckx4- | CAAGGTAAAACTCACACGCCATAACC | CATAAACCCTGGAGCGAAACCTAGAG | GAGCGTCGGTCCCCACACTTCTATAC |
| Z | (SEQ ID No. 24) | (SEQ ID No. 25) | (SEQ ID No. 26) |
| ckx5- | TTGTTGCAGCAACGACCAACCGATAA | AATGGTATATTGTGATGACAGGTGAG | TGGTTCACGTAGTGGGCCATCG |
| S1 | TGA | ATG | (SEQ ID No. 29) |
| | (SEQ ID No. 27) | (SEQ ID No. 28) | |
| ckx5- | AATGGTATATTGTGATGACAGGTGAG | TTGTTGCAGCAACGACCAACCGATAA | ATATTGACCATCATACTCATTGC |
| G2 | ATG | TGA | (SEQ ID No. 32) |
| | (SEQ ID No. 30) | (SEQ ID No. 31) | |
| ckx6- | ACCCTGTCCAAGAATGCTTCA | TGTGGATTCCCCTGCTCCATA | TGGTTCACGTAGTGGGCCATCG |
| S2 | (SEQ ID No. 33) | (SEQ ID No. 34) | (SEQ ID No. 35) |
| ckx7- | TTAGCCGTCCGATCAATCTC | CGGAAAATCTACGGATGGTG | ATATTGACCATCATACTCATTGC |
| G1 | (SEQ ID No. 36) | (SEQ ID No. 37) | (SEQ ID No. 38) |
| ckx7- | GCTAGTAAGTCAGAAGAACGAGTCATC | TTAGCCGTCCGATCAATCTC | GCCTTTTCAGAAATGGATAAATAGCC |
| T1 | (SEQ ID No. 39) | (SEQ ID No. 40) | TTGCTTCC |
| | | | (SEQ ID No. 41) |

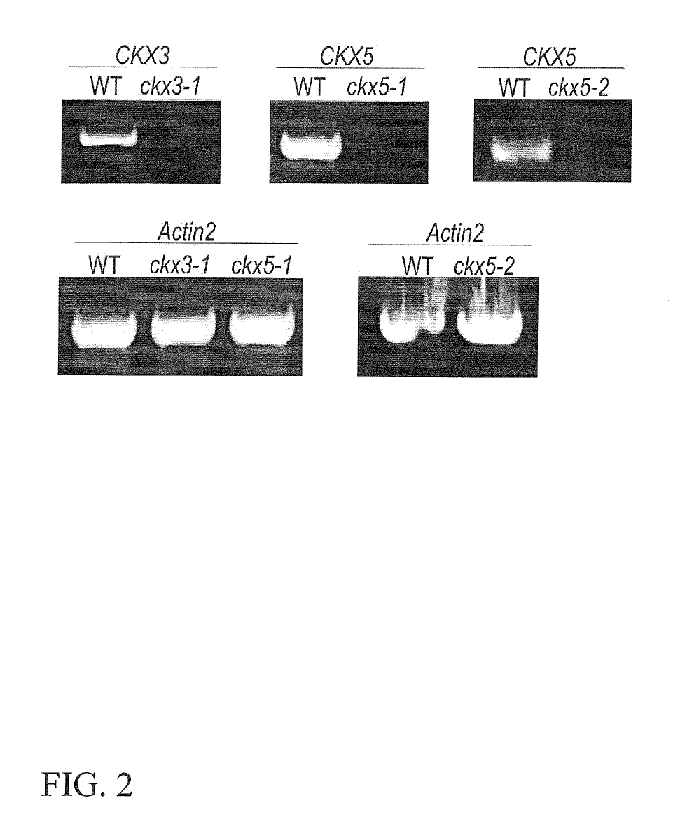

TABLE 2
Primer used for RT-PCR analyses shown in FIG. 2.

| | primer 1 | primer 2 |
|---|---|---|
| ckx2-S1 | GAATGGTGGAATTGGTGGTC | AGTCCCGAAGCTGATTTTTG |
| | (SEQ ID No. 42) | (SEQ ID No. 43) |
| ckx3-S1 | CTCGGCTAAAGACGGAGTTG | AATAGGTGGTTGTAAACGTAGACGCA |
| | (SEQ ID No. 44) | (SEQ ID No. 45) |
| ckx4-S1 | CTCTGCCGCTTCTCACGACTTCGGTA | CATAAACCCTGGAGCGAAACCTAGAG |
| | (SEQ ID No. 46) | (SEQ ID No. 47) |
| ckx4-Z | CTCTGCCGCTTCTCACGACTTCGGTA | CATAAACCCTGGAGCGAAACCTAGAG |
| | (SEQ ID No. 48) | (SEQ ID No. 49) |
| ckx5-S1 | GCACGAATCTCTCTCGAACC | CGCTGACGAAGAAGACGAC |
| | (SEQ ID No. 50) | (SEQ ID No. 51) |
| ckx5-G2 | GCACGAATCTCTCTCGAACC | AAATTCTTGGACCGGAGCTT |
| | (SEQ ID No. 52) | (SEQ ID No. 53) |
| ckx6-S2 | TGTGGATTCCCCTGCTCCATA | ACCCTGTCCAAGAATGCTTCA |
| | (SEQ ID No. 54) | (SEQ ID No. 55) |
| ckx7-G1 | TTAGCCGTCCGATCAATCTC | CGGAAAATCTACGGATGGTG |
| | (SEQ ID No. 56) | (SEQ ID No. 57) |
| ckx7-T1 | TTAGCCGTCCGATCAATCTC | CGGAAAATCTACGGATGGTG |
| | (SEQ ID No. 58) | (SEQ ID No. 59) |
| actin2 | TACAACGAGCTTCGTGTTGC | GATTGATCCTCCGATCCAGA |
| | (SEQ ID No. 60) | (SEQ ID No. 61) |

1. An isolated plant cell comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof; and
ii) one further endogenous gene encoding for a cytokininoxidase/dehydrogenase and being different from the gene defined in i);
wherein said disruptions inhibit expression and/or activity of a product of the at least two disrupted cytokininoxidase/dehydrogenase genes compared to a corresponding control plant cell lacking such disruptions.

2. A transgenic plant comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof; and
ii) one further endogenous gene encoding for a cytokininoxidase/dehydrogenase and being different from the gene defined in i);
wherein said disruptions inhibit expression and/or activity of a product of the at least two disrupted cytokininoxidase/dehydrogenase genes compared to a corresponding control plant lacking such disruptions.

3. An isolated plant cell of claim 1, comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof; and
ii) in at least one further endogenous gene being:
a) a CKX1 gene encoding for a cytokininoxidase/dehydrogenase comprising a polypeptide sequence being identical to or having at least 95% identity with SEQ ID No. 13 or an orthologue thereof;
b) a CKX2 gene encoding for a cytokininoxidase/dehydrogenase comprising a polypeptide sequence being identical to or having at least 95% identity with SEQ ID No. 2 or an orthologue thereof;
c) a CKX4 gene encoding for a cytokininoxidase/dehydrogenase comprising a polypeptide sequence being identical to or having at least 95% identity with SEQ ID No. 3 or an orthologue thereof;
d) a CKX5 gene encoding for a cytokininoxidase/dehydrogenase comprising a polypeptide sequence being identical to or having at least 95% identity with SEQ ID No. 4 or an orthologue thereof;
e) a CKX6 gene encoding for a cytokininoxidase/dehydrogenase comprising a polypeptide sequence being identical to or having at least 95% identity with SEQ ID No. 5 or an orthologue thereof; or
f) a CKX7 gene encoding for a cytokininoxidase/dehydrogenase comprising a polypeptide sequence being identical to or having at least 95% identity with SEQ ID No. 6 or an orthologue thereof.

4. The isolated plant cell of claim 1, wherein at least:
i) an endogenous CKX3 gene comprising a nucleic acid sequence being identical to or having at least 95% identity with SEQ ID No. 7 or an orthologue thereof; and
ii) at least one further endogenous gene being:
a) a CKX1 gene comprising a nucleic acid sequence being identical to or having at least 95% identity with SEQ ID No. 14 or an orthologue thereof;
b) a CKX2 gene comprising a nucleic acid sequence being identical to or having at least 95% identity with SEQ ID No. 8 or an orthologue thereof;
c) a CKX4 gene comprising a nucleic acid sequence being identical to or having at least 95% identity with SEQ ID No. 9 or an orthologue thereof;
d) a CKX5 gene comprising a nucleic acid sequence being identical to or having at least 95% identity with SEQ ID No. 10 or an orthologue thereof;
e) a CKX6 gene comprising a nucleic acid sequence being identical to or having at least 95% identity with SEQ ID No. 11 or an orthologue thereof; or
f) a CKX7 gene comprising a nucleic acid sequence being identical to or having at least 95% identity with SEQ ID No. 12 or an orthologue thereof;
are disrupted.

5. The isolated plant cell of claim 1, wherein i) at least an endogenous CKX3 gene encoding for a cytokininoxidase/dehydrogenase comprising a polypeptide sequence being identical to or having at least 95% identity with SEQ ID No. 1 or an orthologue thereof; and
ii) an endogenous CKX5 gene encoding for a cytokininoxidase/dehydrogenase comprising a polypeptide sequence being identical to or having at least 95% identity with SEQ ID No. 4 or an orthologue thereof, are disrupted.

6. The isolated plant cell of claim 4, wherein i) an endogenous CKX3 gene comprising a nucleic acid sequence being identical to or having at least 95% identity with SEQ ID No. 7 or an orthologue thereof; and
ii) an endogenous CKX5 gene comprising a nucleic acid sequence being identical to or having at least 95% identity with SEQ ID No. 10 or an orthologue thereof; are disrupted.

7. The isolated plant cell of claim 1, wherein one, more than one or all disruptions are facilitated by structural disruption, antisense polynucleotide gene suppression, double stranded RNA induced gene silencing, ribozyme techniques, genomic disruptions, tilling, and/or homologous recombination.

8. The isolated plant cell of claim 1, wherein one, more than one or all disruptions are homozygous disruptions.

9-15. (canceled)

16. A transgenic plant of claim 2, comprising a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof; and
ii) in at least one further endogenous gene being:
a) a CKX1 gene encoding for a cytokininoxidase/dehydrogenase comprising a polypeptide sequence being identical to or having at least 95% identity with SEQ ID No. 13 or an orthologue thereof;
b) a CKX2 gene encoding for a cytokininoxidase/dehydrogenase comprising a polypeptide sequence being identical to or having at least 95% identity with SEQ ID No. 2 or an orthologue thereof;
c) a CKX4 gene encoding for a cytokininoxidase/dehydrogenase comprising a polypeptide sequence being identical to or having at least 95% identity with SEQ ID No. 3 or an orthologue thereof;
d) a CKX5 gene encoding for a cytokininoxidase/dehydrogenase comprising a polypeptide sequence being identical to or having at least 95% identity with SEQ ID No. 4 or an orthologue thereof;
e) a CKX6 gene encoding for a cytokininoxidase/dehydrogenase comprising a polypeptide sequence being identical to or having at least 95% identity with SEQ ID No. 5 or an orthologue thereof; or
f) a CKX7 gene encoding for a cytokininoxidase/dehydrogenase comprising a polypeptide sequence being identical to or having at least 95% identity with SEQ ID No. 6 or an orthologue thereof.

17. The transgenic plant of claim 2, wherein at least:
i) an endogenous CKX3 gene comprising a nucleic acid sequence being identical to or having at least 95% identity with SEQ ID No. 7 or an orthologue thereof; and
ii) at least one further endogenous gene being:
a) a CKX1 gene comprising a nucleic acid sequence being identical to or having at least 95% identity with SEQ ID No. 14 or an orthologue thereof;
b) a CKX2 gene comprising a nucleic acid sequence being identical to or having at least 95% identity with SEQ ID No. 8 or an orthologue thereof;
c) a CKX4 gene comprising a nucleic acid sequence being identical to or having at least 95% identity with SEQ ID No. 9 or an orthologue thereof;
d) a CKX5 gene comprising a nucleic acid sequence being identical to or having at least 95% identity with SEQ ID No. 10 or an orthologue thereof;
e) a CKX6 gene comprising a nucleic acid sequence being identical to or having at least 95% identity with SEQ ID No. 11 or an orthologue thereof; or
f) a CKX7 gene comprising a nucleic acid sequence being identical to or having at least 95% identity with SEQ ID No. 12 or an orthologue thereof;
are disrupted.

18. The transgenic plant of claim 2, wherein i) at least an endogenous CKX3 gene encoding for a cytokininoxidase/dehydrogenase comprising a polypeptide sequence being identical to or having at least 95% identity with SEQ ID No. 1 or an orthologue thereof; and
ii) an endogenous CKX5 gene encoding for a cytokininoxidase/dehydrogenase comprising a polypeptide sequence being identical to or having at least 95% identity with SEQ ID No. 4 or an orthologue thereof, are disrupted.

19. The transgenic plant of claim 17, wherein i) an endogenous CKX3 gene comprising a nucleic acid sequence being identical to or having at least 95% identity with SEQ ID No. 7 or an orthologue thereof; and
ii) an endogenous CKX5 gene comprising a nucleic acid sequence being identical to or having at least 95% identity with SEQ ID No. 10 or an orthologue thereof; are disrupted.

20. The transgenic plant of claim 2, wherein one, more than one or all disruptions are facilitated by structural disruption, antisense polynucleotide gene suppression, double stranded RNA induced gene silencing, ribozyme techniques, genomic disruptions, tilling, and/or homologous recombination.

21. The transgenic plant of claim 2, wherein one, more than one or all disruptions are homozygous disruptions.

22. The transgenic plant of claim 2, wherein the plant is selected from the family Brassicaceae, preferably from the genera Brassica or Arabidopsis.

23. A cell, organ, tissue or transgenic propagation material derived from the transgenic plant of claim 2.

24. A method of increasing a seed yield in a plant and/or increasing plant height and/or increasing stem thickness relative to a corresponding control plant, the method comprising introducing in a plant a disruption in at least:
i) an endogenous CKX3 gene encoding for a cytokininoxidase/dehydrogenase comprising a polypeptide sequence being identical to or having at least 95% identity with SEQ ID No. 1 or an orthologue thereof; and
ii) one further endogenous gene encoding for a cytokininoxidase/dehydrogenase and being different from the gene defined in i);
wherein said disruptions inhibit expression and/or activity of a product of the at least two disrupted cytokininoxidase/dehydrogenase genes compared to a corresponding control plant lacking such disruptions.

25. A method for producing a plant with an increased seed yield and/or plant height relative to a corresponding control plant, comprising disrupting in a plant at least:
i) an endogenous CKX3 gene encoding for a cytokininoxidase/dehydrogenase comprising a polypeptide sequence being identical to or having at least 95% identity with SEQ ID No. 1 or an orthologue thereof; and
ii) one further endogenous gene encoding for a cytokininoxidase/dehydrogenase and being different from the gene defined in i);
wherein said disruptions inhibit expression and/or activity of a product of the at least two disrupted cytokininoxidase/dehydrogenase genes compared to a corresponding control plant lacking such disruptions.

26. The method of claim 24, wherein at least
i) an endogenous CKX3 gene encoding for a cytokininoxidase/dehydrogenase comprising a polypeptide sequence being identical to or having at least 95% identity with SEQ ID No. 1 or an orthologue thereof; and
ii) an endogenous CKX5 gene encoding for a cytokininoxidase/dehydrogenase comprising a polypeptide sequence being identical to or having at least 95% identity with SEQ ID No. 4 or an orthologue thereof, are disrupted.

27. The method of claim 25, wherein at least
i) an endogenous CKX3 gene encoding for a cytokininoxidase/dehydrogenase comprising a polypeptide sequence being identical to or having at least 95% identity with SEQ ID No. 1 or an orthologue thereof; and
ii) an endogenous CKX5 gene encoding for a cytokininoxidase/dehydrogenase comprising a polypeptide sequence being identical to or having at least 95% identity with SEQ ID No. 4 or an orthologue thereof, are disrupted.

28. The method of claim 24, wherein one, more than one or all disruptions are homozygous disruptions.

29. The method of claim 25, wherein one, more than one or all disruptions are homozygous disruptions.

30. A transgenic plant obtainable or obtained by the method of claim 24.

31. A transgenic plant obtainable or obtained by the method of claim 25.


SEQUENCE LISTING (61 sequences)

SEQ ID NO: 1 - protein, 523 aa, Arabidopsis thaliana
MASYNLRSQVRLIAITIVIIITLSTPITTNTSPQPWNILSHNEFAGKLTSSSSSVESAATDFGHVTKIFPSAVLIPSSVEDITDLIKLSFDSQLSFPLAARGHGHSHRGQASAKDGVVVNMRSMVNRDRGIKVSRTCLYVDVDAAWLWIEVLNKTLELGLTPVSWTDYLYLTVGGTLSNGGISGQTFRYGPQITNVLEMDVITGKGEIATCSKDMNSDLFFAVLGGLGQFGIITRARIKLEVAPKRAKWLRFLYIDFSEFTRDQERVISKTDGVDFLEGSIMVDHGPPDNWRSTYYPPSDHLRIASMVKRHRVIYCLEVVKYYDETSQYTVNEEMEELSDSLNHVRGFMYEKDVTYMDFLNRVRTGELNLKSKGQWDVPHPWLNLFVPKTQISKFDDGVFKGIILRNNITSGPVLVYPMNRNKWNDRMSAAIPEEDVFYAVGFLRSAGFDNWEAFDQENMEILKFCEDANMGVIQYLPYHSSQEGWVRHFGPRWNIFVERKYKYDPKMILSPGQNIFQKINSS

SEQ ID NO: 2 - protein, 501 aa, Arabidopsis thaliana
MANLRLMITLITVLMITKSSNGIKIDLPKSLNLTLSTDPSIISAASHDFGNITTVTPGGVICPSSTADISRLLQYAANGKSTFQVAARGQGHSLNGQASVSGGVIVNMTCITDVVVSKDKKYADVAAGTLWVDVLKKTAEKGVSPVSWTDYLHITVGGTLSNGGIGGQVFRNGPLVSNVLELDVITGKGEMLTCSRQLNPELFYGVLGGLGQFGIITRARIVLDHAPKRAKWFRMLYSDFTTFTKDQERLISMANDIGVDYLEGQIFLSNGVVDTSFFPPSDQSKVADLVKQHGIIYVLEVAKYYDDPNLPIISKVIDTLTKTLSYLPGFISMHDVAYFDFLNRVHVEENKLRSLGLWELPHPWLNLYVPKSRILDFHNGVVKDILLKQKSASGLALLYPTNRNKWDNRMSAMIPEIDEDVIYIIGLLQSATPKDLPEVESVNEKIIRFCKDSGIKIKQYLMHYTSKEDWIEHFGSKWDDFSKRKDLFDPKKLLSPGQDIF

SEQ ID NO: 3 - protein, 524 aa, Arabidopsis thaliana
MTNTLCLSLITLITLFISLTPTLIKSDEGIDVFLPISLNLTVLTDPFSISAASHDFGNITDENPGAVLCPSSTTEVARLLRFANGGFSYNKGSTSPASTFKVAARGQGHSLRGQASAPGGVVVNMTCLAMAAKPAAVVISADGTYADVAAGTMWVDVLKAAVDRGVSPVTWTDYLYLSVGGTLSNAGIGGQTFRHGPQISNVHELDVITGKGEMMTCSPKLNPELFYGVLGGLGQFGIITRARIALDHAPTRVKWSRILYSDFSAFKRDQERLISMTNDLGVDFLEGQLMMSNGFVDTSFFPLSDQTRVASLVNDHRIIYVLEVAKYYDRTTLPIIDQVIDTLSRTLGFAPGFMFVQDVPYFDFLNRVRNEEDKLRSLGLWEVPHPWLNIFVPGSRIQDFHDGVINGLLLNQTSTSGVTLFYPTNRNKWNNRMSTMTPDEDVFYVIGLLQSAGGSQNWQELENLNDKVIQFCENSGIKIKEYLMHYTRKEDWVKHFGPKWDDFLRKKIMFDPKRLLSPGQDIFN

SEQ ID NO: 4 - protein, 540 aa, Arabidopsis thaliana
MNREMTSSFLLLTFAICKLIIAVGLNVGPSELLRIGAIDVDGHFTVHPSDLASVSSDFGMLKSPEEPLAVLHPSSAEDVARLVRTAYGSATAFPVSARGHGHSINGQAAAGRNGVVVEMNHGVTGTPKPLVRPDEMYVDVWGGELWVDVLKKTLEHGLAPKSWTDYLYLTVGGTLSNAGISGQAFHHGPQISNVLELDVVTGKGEVMRCSEEENTRLFHGVLGGLGQFGIITRARISLEPAPQRVRWIRVLYSSFKVFTEDQEYLISMHGQLKFDYVEGFVIVDEGLVNNWRSSFFSPRNPVKISSVSSNGSVLYCLEITKNYHDSDSEIVDQEVEILMKKLNFIPTSVFTTDLQYVDFLDRVHKAELKLRSKNLWEVPHPWLNLFVPKSRISDFDKGVFKGILGNKTSGPILIYPMNKDKWDERSSAVTPDEEVFYLVALLRSALTDGEETQKLEYLKDQNRRILEFCEQAKINVKQYLPHHATQEEWVAHFGDKWDRFRSLKAEFDPRHILATGQRIFQNPSLSLFPPSSSSSSAASW

SEQ ID NO: 5 - protein, 533 aa, Arabidopsis thaliana
MSYLHASLLRKRTMLIVRSFTILLLSCIAFKLACCFSSSISSLKALPLVGHLEFEHVHHASKDFGNRYQLIPLAVLHPKSVSDIASTIRHIWMMGTHSQLTVAARGRGHSLQGQAQTRHGIVIHMESLHPQKLQVYSVDSPAPYVDVSGGELWINILHETLKYGLAPKSWTDYLHLTVGGTLSNAGISGQAFRHGPQISNVHQLEIVTGKGEILNCTKRQNSDLFNGVLGGLGQFGIITRARIALEPAPTMVKWIRVLYLDFAAFAKDQEQLISAQGHKFDYIEGFVIINRTGLLNSWRLSFTAEEPLEASQFKFDGRTLYCLELAKYLKQDNKDVINQEVKETLSELSYVTSTLFTTEVAYEAFLDRVHVSEVKLRSKGQWEVPHPWLNLLVPRSKINEFARGVFGNILTDTSNGPVIVYPVNKSKWDNQTSAVTPEEEVFYLVAILTSASPGSAGKDGVEEILRRNRRILEFSEEAGIGLKQYLPHYTTREEWRSHFGDKWGEFVRRKSRYDPLAILAPGHRIFQKAVSYS

SEQ ID NO: 6 - protein, 524 aa, Arabidopsis thaliana
MIAYIEPYFLENDAEAASAATAAGKSTDGVSESLNIQGEILCGGAAADIAGRDFGGMNCVKPLAVVRPVGPEDIAGAVKAALRSDKLTVAARGNGHSINGQAMAEGGLVVDMSTTAENHFEVGYLSGGDATAFVDVSGGALWEDVLKRCVSEYGLAPRSWTDYLGLTVGGTLSNAGVSGQAFRYGPQTSNVTELDVVTGNGDVVTCSEIENSELFFSVLGGLGQFGIITRARVLLQPAPDMVRWIRVVYTEFDEFTQDAEWLVSQKNESSFDYVEGFVFVNGADPVNGWPTVPLHPDHEFDPTRLPQSCGSVLYCLELGLHYRDSDSNSTIDKRVERLIGRLRFNEGLRFEVDLPYVDFLLRVKRSEEIAKENGTWETPHPWLNLFVSKRDIGDFNRTVFKELVKNGVNGPMLVYPLLRSRWDDRTSVVIPEEGEIFYIVALLRFVPPCAKVSSVEKMVAQNQEIVHWCVKNGIDYKLYLPHYKSQEEWIRHFGNRWSRFVDRKAMFDPMAILSPGQKIFNRSL

SEQ ID NO: 7 - DNA, 3342 bp, Arabidopsis thaliana
atggcgagttataatcttcgttcacaagttcgtcttatagcaataacaatagtaatcatcattactctctcaactccgatcacaaccaacacatcaccacaaccatggaatatcctttcacacaacgaattcgccggaaaactcacctcctcctcctcctccgtcgaatcagccgccacagatttcggccacgtcaccaaaatcttcccttccgccgtcttaatcccttcctccgttgaagacatcacagatctcataaaactctcttttgactctcaactgtcttttcctttagccgctcgtggtcacggacacagccaccgtggccaagcctcggctaaagacggagttgtggtcaacatgcggtccatggtaaaccgggatcgaggtatcaaggtgtctaggacctgtttatatgttgacgtggacgctgcgtggctatggattgaggtgttgaataaaactttggagttagggttaacgccggtttcttggacggattatttgtatttaacagtcggtgggacgttatcaaacggcggaattagtggacaaacgtttcggtacggtccacagatcactaatgttctagagatggatgttattactggtacgtaccacgatctttttcacacagagattaaaaaaaacagtaatagtgattttaacttcgtacgtttctgatagacaacaaagaacttcgtacgtttttcgaagttttttcgtctttttcattttagatctgcgcggccatttttggttatgctattgtttgtttgtattgtttgtctctgtttatttatttctcgaacttgttgatagcttttcttcttttcacacatcaatctaatcaccttttttggtcttaagattagaaagaagatacggactaggtaaaaataggtggttgtaaacgtagacgcattaaaaaaatattggtttttttattttttgataagcaaaattggtggttggtctaagattataaacttgatattaatgcaaaggtcgatctagcaatagaagattaatcaatattcttggtgttttaacaacagattatttcatcattaaaatcgtgaaacaaagaaattttggtagtatacattacgtgtagttttgttagtttattaaaaaaaatagtatatagttttgttaaaacgcgatttatttagtaacacattagtatattacacgtttaaccaactaaacttttttttttgaataattatgttctatatttcttactcaaattatgcaaatttcgtggattcgaagtcaaatttctgcgaaatttacatggtcatatattataaaactgttcatataacccggtgaacaaacagacaattaagggtttgaatggttacggcggttggggcggacacaaccgtcaatagatcagaccgttttttatttaccattcatcaattatattccgcagtggtttggggtaaaaaaaatagaagaaaaccgcagcggaccaattccataccgtttttacatacaaataaacatggtgcgcaacggtttattgtccgcctcaaaaatgaaatggactaaaccgcagataaattagaccgctttgtccgctgcctccattcatagactaaaaaaaaacaaccaaaaaaaaaatggtcccacgcccatgattttacacgaggtttcttgtggcgtaaggacaaaactcaaaagttcataacgtttggtcctaaccaggtgtaatggattaagtaacagtcaattttcttattatagctgtatccattatgtccacatatgcatccatatacattacactgttggtctcaagtgtagttagattacgaagactttcaagttccattttttggttaggagataaacataatttaatgataccgactttagcactctaggctcaaaacaagtacagaagagaatagttttatttcaaactcgttgcattgttgtatcaattaattgtgttagtctttgtatattcttacataacggtccaagtttgttgaaatagtttacttactaaacttttcctaatggggtcaaattttattttataggaaaaggagagattgcaacttgttccaaggacatgaactcggatcttttcttcgcggtgttaggaggtttgggtcaattcggcattataacaagagccagaattaaacttgaagtagctccgaaaagggtatgttaaatttgtaaattatgcaactacagaaaattctatgaaatttatgaatgaacatatatgcatttttggatttttgtaggccaagtggttaaggtttctatacatagatttctccgaattcacaagagatcaagaacgagtgatatcgaaaacggacggtgtagatttcttagaaggttccattatggtggaccatggcccaccggataactggagatccacgtattatccaccgtccgatcacttgaggatcgcctcaatggtcaaacgacatcgtgtcatctactgccttgaagtcgtcaagtattacgacgaaacttctcaatacacagtcaacgaggtccgtacatacatacaatcataaatcatacatgtataattgggagatctttatgcattattcaattatattaatttactttagttatttaacttatgcaggaaatggaggagttaagcgatagtttaaaccatgtaagagggtttatgtacgagaaagatgtgacgtatatggatttcctaaaccgagttcgaaccggagagctaaacctgaaatccaaaggccaatgggatgttccacatccatggcttaatctcttcgtaccaaaaactcaaatctccaaatttgatgatggtgtttttaagggtattatcctaagaaataacatcactagcggtcctgttcttgtttatcctatgaatcgcaacaagtaagtttaactcgatattgcaaaatttactatctacattttcgttttggaatccgaaatattcttacaagctaattttatgcggcgtttttaggtggaatgatcggatgtctgccgctatacccgaggaagatgtattttatgcggtagggtttttaagatccgcgggttttgacaattgggaggcttttgatcaagaaaacatggaaatactgaagttttgtgaggatgctaatatgggggttatacaatatcttccttatcattcatcacaagaaggatgggttagacattttggtccgaggtggaatattttcgtagagagaaaatataaatatgatcccaaaatgatattatcaccgggacaaaatatatttcaaaaaataaactcgagttagacgataattaatcctattgttagtcgttctcaccactttg

SEQ ID NO: 8 - DNA, 2991 bp, Arabidopsis thaliana
atggctaatcttcgtttaatgatcactttaatcacggttttaatgatcaccaaatcatcaaacggtattaaaattgatttacctaaatcccttaacctcaccctctctaccgatccttccatcatctccgcagcctctcatgacttcggaaacataaccaccgtgacccccggcggcgtaatctgcccctcctccaccgctgatatctctcgtctcctccaatacgccgcaaacggaaaaagtacattccaagtagcggctcgtggccaaggccactccttaaacggccaagcctcggtctccggcggagtaatcgtcaacatgacgtgtatcactgacgtggtggtttcaaaagacaagaagtacgctgacgtggcggccgggacgttatgggtggatgtgcttaagaagacggcggagaaaggggtgtcgccggtttcttggacggattatttgcatataaccgtcggaggaacgttgtcgaatggtggaattggtggtcaagtgtttcgaaacggtcctcttgttagtaacgtccttgaattggacgttattactggtacgcatcttctaaactttgatgtacatacaacaacaaaaactgtttttgttttatagtatttttcattttttgtaccataggttttatgttttatagttgtgctaaacttcttgcaccacacgtaagtcttcgaaacacaaaatgcgtaacgcatctatatgttttttgtacatattgaatgttgttcatgagaaataaagtaattacatatacacacatttattgtcgtacatatataaataattaaagacaaattttcacaattggtagcgtgttaatttgggatttttgtaatgtacatgcatgacgcatgcatatggagcttttcggttttcttagatttgtgtagtatttcaaatatatcatttattttctttcgaataaagaggtggtatatttttaaaatagcaacatttcagaatttttctttgaatttacactttttaaattgttattgttaatatggattttgaataaataatttcagggaaaggtgaaatgttgacatgctcgcgacagctaaacccagaattgttctatggagtgttaggaggtttgggtcaatttggaattataacgagagccagaattgttttggaccatgcacctaaacgggtacgtatcatcatattttaccatttgttttagtcagcattcatttttcattagtaattccgtttcaatttctaaatttttttagtcaatagaaaatgattcttatgtcagagcttgattatttagtgatttttattgagataaaataaaatataacctaacggaaataattattttactaatcggataatgtctgattaaaacattttatgatattacactaagagagttagagacgtatggatcacaaaacatgaagctttcttagatggtatcctaaaactaaagttaggtacaagtttggaatttaggtcaaatgcttaagttgcattaatttgaacaaaatctatgcattgaataaaaaaaagatatggattattttataaagtatagtccttgtaatcctaggacttgttgtctaatcttgtcttatgcgtgcaaatctttttgatgtcaatatataatccttgtttattagagtcaagctctttcattagtcaactactcaaatatactccaaagtttagaatatagtcttctgactaattagaatcttacaaccgataaacgttacaatttggttatcattttaaaaaacagatttggtcataatatacgatgacgttctgttttagtttcatctattcacaaattttatataattattttcaagaaaatattgaaatactatactgtaatatggtttctttatatatgtgtgtataaattaaatgggattgttttctctaaatgaaattgtgtaggccaaatggtttcggatgctctacagtgatttcacaacttttacaaaggaccaagaacgtttgatatcaatggcaaacgatattggagtcgactatttagaaggtcaaatatttctatcaaacggtgtcgttgacacctcttttttcccaccttcagatcaatctaaagtcgctgatctagtcaagcaacacggtatcatctatgttcttgaagtagccaagtattatgatgatcccaatctccccatcatcagcaaggtactacacatttacattttcatcatcgtttttatcataccataagatatttaaatgattcatcattgcaccacattaagatattcatcatcatcatcgttacatttttttttgcatcttatgcttctcataatctactattgtgtaggttattgacacattaacgaaaacattaagttacttgcccgggttcatatcaatgcacgacgtggcctacttcgatttcttgaaccgtgtacatgtcgaagaaaataaactcagatctttgggattatgggaacttcctcatccttggcttaacctctacgttcctaaatctcggattctcgattttcataacggtgttgtcaaagacattcttcttaagcaaaaatcagcttcgggactcgctcttctctatccaacaaaccggaataagtacatacttctcttcattcatatttatcttcaagaaccaaagtaaataaatttctatgaactgattatgctgttattgttagatgggacaatcgtatgtcggcgatgataccagagatcgatgaagatgttatatatattatcggactactacaatccgctaccccaaaggatcttccagaagtggagagcgttaacgagaagataattaggttttgcaaggattcaggtattaagattaagcaatatctaatgcattatactagtaaagaagattggattgagcattttggatcaaaatgggatgatttttcgaagaggaaagatctatttgatcccaagaaactgttatctccagggcaagacatcttttga

SEQ ID NO: 9 - DNA, 2782 bp, Arabidopsis thaliana
atgactaatactctctgtttaagcctcatcaccctaataacgctttttataagtttaaccccaaccttaatcaaatcagatgagggcattgatgttttcttacccatatcactcaaccttacggtcctaaccgatcccttctccatctctgccgcttctcacgacttcggtaacataaccgacgaaaatcccggcgccgtcctctgcccttcctccaccacggaggtggctcgtctcctccgtttcgctaacggaggattctcttacaataaaggctcaaccagccccgcgtctactttcaaagtggctgctcgaggccaaggccactccctccgtggccaagcctctgcacccggaggtgtcgtcgtgaacatgacgtgtctcgccatggcggctaaaccagcggcggttgttatctcggcagacgggacttacgctgacgtggctgccgggacgatgtgggtggatgttctgaaggcggcggtggatagaggcgtctcgccggttacatggacggattatttgtatctcagcgtcggcgggacgttgtcgaacgctggaatcggtggtcagacgtttagacacggccctcagattagtaacgttcatgagcttgacgttattaccggtacgtaaataccaaaacttcactaatctcgttacaattttttaattttttggtaatataaattttgtacggctcaactcttaattaagaatgaaacagtatctatgatcttctagatgctctttttttgtctgcaagctttaattgtagtaacatcagcgatatatatatcacatgcatgtgtattattgatgataatatataatgttttagttacaaatttgattctcaaggtaaaactcacacgccataaccagtataaaactccaaaaatcacgttttggtcagaaatacatatccttcattaacagtagttatgctataatttgtgattataaataactccggagtttgttcacaatactaaatttcaggaaaaggtgaaatgatgacttgctctccaaagttaaaccctgaattgttctatggagttttaggaggtttgggtcaattcggtattataacgagggccaggattgcgttggatcatgcacccacaagggtatgtatcatgcatctatagtgtaatcaatttataattttaatgtagtggtcctaaatccaaaatttgatttgatttggttggaacgtacgtatatataataagtcaaaaggctgattttgaagacgaatttatatacttttgttgaattaaatctgattttgcttacgttttattagattctgcgtaataaatcctaggacttgctcgagtgtaatcttgtcttatgcttgcaaatcttgttgatgtcaatatctaatcttttttattatatttccctacgtaagttttagatatagttattttaaactgctataaattgtgtacgtatagactttagataaaaagttgtggtcgcttgcacctatttgtttatcgctatagtgattcaaaggtctatatatgattcttggtttttctttttgaaaaaaatagaccatacaatccaaggaagatgatcttaaatggactaatttatggatataaattgatatacaaatctgcaggtgaaatggtctcgcatactctacagtgacttctcggcttttaaaagagaccaagagcgtttaatatcaatgaccaatgatctcggagttgactttttggaaggtcaacttatgatgtcaaatggcttcgtagacacctctttcttcccactctccgatcaaacaagagtcgcatctcttgtgaatgaccaccggatcatctatgttctcgaagtagccaagtattatgacagaaccacccttcccattattgaccaggtactaaaatccattattcatgatgattatcttcacacaatcagtatcatcaccaattaccatcatcacttgtcatatatgatccaaagtaaatatatcacatgatataaataaatcgttcaaatctttttttttaaagaataaaagaatcattttcaagcattactcatacacatctacgaatcaccgtgaccatatataaccatacgcttattaaataatcatttttgtttgtaggtgattgacacgttaagtagaactctaggtttcgctccagggtttatgttcgtacaagatgttccgtatttcgatttcttgaaccgtgtccgaaacgaagaagataaactcagatctttaggactatgggaagttcctcatccatggcttaacatctttgtcccggggtctcgaatccaagattttcatgatggtgttattaatggccttcttctaaaccaaacctcaacttctggtgttactctcttctatcccacaaaccgaaacaagtaaatatttactttttgattttgttttatttgaaagtatatcccaataatgtatgttaaattgttaacaagaatttattttattaatagatggaacaaccgcatgtcaacgatgacaccggacgaagatgttttttatgtgatcggattactgcaatcagctggtggatctcaaaattggcaagaacttgaaaatctcaacgacaaggttattcagttttgtgaaaactcgggaattaagattaaggaatatttgatgcactatacaagaaaagaagattgggttaaacattttggaccaaaatgggatgattttttaagaaagaaaattatgtttgatcccaaaagactattgtctccaggacaagacatatttaattaa

SEQ ID NO: 10 - DNA, 2817 bp, Arabidopsis thaliana
atgaatcgtgaaatgacgtcaagctttcttctcctgacgttcgccatatgtaaactgatcatagccgtgggtctaaacgtgggccccagtgagctcctccgcatcggagccatagatgtcgacggccacttcaccgtccacccttccgacttagcctccgtctcctcagacttcggtatgctgaagtcacctgaagagccattggccgtgcttcatccatcatcggccgaagacgtggcacgactcgtcagaacagcttacggttcagccacggcgtttccggtctcagcccgaggccacggccattccataaacggacaagccgcggcggggaggaacggtgtggtggttgaaatgaaccacggcgtaaccgggacgcccaagccactcgtccgaccggatgaaatgtatgtggatgtatggggtggagagttatgggtcgatgtgttgaagaaaacgttggagcatggcttagcaccaaaatcatggacggattacttgtatctaaccgttggaggtacactctccaatgcaggaatcagtggtcaagcttttcaccatggtcctcaaattagtaacgtccttgagctcgacgttgtaactggttagtattaaaacattcaagttcatatattttaaatgcttttgtctgaagttttactaataacaagaaattgataccaaaaagtagggaaaggagaggtgatgagatgctcagaagaagagaacacaaggctattccatggagttcttggtggattaggtcaatttgggatcatcactcgagcacgaatctctctcgaaccagctccccaaagggtaatatttttttaatgactagctatcaaaaatccctggcgggtccatacgttgtaatctttttagtttttactgttgatggtattttttatatattttggataataaaaccctaaaatggtatattgtgatgacaggtgagatggatacgggtattgtattcgagcttcaaagtgtttacggaggaccaagagtacttaatctcaatgcatggtcaattaaagtttgattacgtggaaggttttgtgattgtggacgaaggactcgtcaacaattggagatcttctttcttctctccacgtaaccccgtcaagatctcctctgttagttccaacggctctgttttgtattgccttgagatcaccaagaactaccacgactccgactccgaaatcgttgatcaggtcactttcattattcacttagaaaaaagcgatattttcattttttatattgatgaatatctggaaggatttaacgctatgcgactattgggaaatcattatgaaaaaatatttagtttatatgattgaaagtggtctccatagtatttttgttgtgtcgactttattataacttaaatttggaagaggacatgaagaagaagccagagaggatctacagagatctagcttttccacctgaacttaataatgcacatttatataattatttttcttcttctaaagtttagtttatcactagcgaattaatcatggttactaattaagtagtggacagggtcatggaccactcactcaccaaataatgattcctctttactcttaagtttaattttaataaaaccaactctactggaatcttaacttatccttggttttggtaggcttttatagcaacacggtttttttaattttcctattccagattttgtatattaaatgtcgattttttttctttttgtttcaggaagttgagattctgatgaagaaattgaatttcataccgacatcggtctttacaacggatttacaatatgtggactttctcgaccgggtacacaaggccgaattgaagctccggtccaagaatttatgggaggttccacacccatggctcaacctcttcgtgccaaaatcaagaatctctgacttcgataaaggcgttttcaagggcattttgggaaataaaacaagtggccctattcttatctaccccatgaacaaagacaagtaagtcttgacattaccattgattactacttctaaatttcttctctagaaaaaagaataaaacgagttttgcattgcatgcatgcaaagttacacttgtggggattaattagtggtccaagaaaaaaagtttgtcaaaattgaaaaaaactagacacgtggtacatgggattgtccgaaaaacgttgtccacatgtgcatcgaaccagctaagattgacaacaacacttcgtcggctcgtatttctctttttgttttgtgaccaaatccgatggtccagattgggtttatttgtttttaagttcctagaactcatggtgggtgggtcccaatcagattctcctagaccaaaccgatctcaacgaaccctccgcacatcattgattattacattaatatagatattgtcgttgctgacgtgtcgtaatttgatgttattgtcagatgggacgagaggagctcagccgtgacgccggatgaggaagttttctatctggtggctctattgagatcagctttaacggacggtgaagagacacagaagctagagtatctgaaagatcagaaccgtcggatcttggagttctgtgaacaagccaagatcaatgtgaagcagtatcttcctcaccacgcaacacaggaagagtgggtggctcattttggggacaagtgggatcggttcagaagcttaaaggctgagtttgatccgcgacacatactcgctactggtcagagaatctttcaaaacccatctttgtctttgtttcctccgtcgtcgtcttcttcgtcagcggcttcatggtga

SEQ ID NO: 11 - DNA, 1975 bp, Arabidopsis thaliana
atgagctatctacatgcaagcctcctcaggaaaagaaccatgcttatagtaagaagtttcaccatcttgcttctcagctgcatagcctttaagttggcttgctgcttctctagcagcatttcttctttgaaggcgcttcccctagtaggccatttggagtttgaacatgtccatcacgcctccaaagattttggaaatcgataccagttgatccctttggcggtcttacatcccaaatcggtaagcgacatcgcctcaacgatacgacacatctggatgatgggcactcattcacagcttacagtggcagcgagaggtcgtggacattcactccaaggccaagctcaaacaagacatggaattgttatacacatggaatcactccatccccagaagctgcaggtctacagtgtggattcccctgctccatatgttgatgtgtctggtggtgagctgtggataaacattttgcatgagaccctcaagtacgggcttgcaccaaaatcatggacggattacctgcatttaactgtaggtggtactctgtccaatgctggaataagcggccaggcattccgacatggaccacagatcagcaatgttcatcaactggagattgtcacaggttagttcagagttgcagtattcgtgttttgaaagcatagactctatatggttggtgactattaacaacatgaagagattcccgagaatagctacccactaatgtcatgcctatttattgactgcaggaaaaggcgagatcctaaactgtacaaagaggcagaacagcgacttatttaatggtgttcttggtggtttaggtcagtttggcatcataacgcgggcaagaatagcattggaaccagcaccaaccatggtaaacaataaataaataaaaaacttaaaaactgaacacgcgtgtgtcctcctaactctgtataatggacaggtaaaatggataagagtgttatacctggattttgcagcttttgccaaggaccaagagcaactaatatctgcccagggccacaaattcgattacatagaagggtttgtgataataaacaggacaggcctcctgaacagctggaggttgtctttcaccgcagaagagcctttagaagcaagccaattcaagtttgatggaaggactctgtattgtctggagctagccaagtatttgaagcaagataacaaagacgtaatcaaccaggtgagaaaacagagtagaagcaatcggtagaatcttctttggtagatgacattcattggaactgaaaatatatatatatttgtccaatccaggaagtgaaagaaacattatcagagctaagctacgtgacgtcgacactgtttacaacggaggtagcatatgaagcattcttggacagggtacatgtgtctgaggtaaaactccgatcgaaagggcagtgggaggtgccacatccatggctgaacctcctggtaccaagaagcaaaatcaatgaatttgcaagaggtgtatttggaaacatactaacggatacaagcaacggcccagtcatcgtctacccagtgaacaaatcaaagtaagaaagaaagaaagaaagagctagtcatgattttgtttcttttcacttgttgacaaaacaaaagcatgttggtgagcaggtgggacaatcaaacatcagcagtaacaccggaggaagaggtattctacctggtggcgatcctaacatcggcatctccagggtcggcaggaaaggatggagtagaagagatcttgaggcggaacagaagaatactggaattcagtgaagaagcagggatagggttgaagcagtatctgccacattacacgacaagagaagagtggagatcccatttcggggacaagtggggagaatttgtgaggaggaaatccagatatgatccattggcaattcttgcgcctggccaccgaatttttcaaaaggcagtctcatactcatga

SEQ ID NO: 12 - DNA, 3211 bp, Arabidopsis thaliana
atgatagcttacatagaaccatacttcttggaaaacgacgccgaggctgcctctgccgccaccgccgccggaaaatctacggatggtgtttctgagtcacttaacatccaaggagaaatcttatgtggtggagctgcggcggatatcgccgggagagattttggcggcatgaactgtgtgaagcctcttgctgtggtgagaccagtgggaccggaagatatcgccggagcggtgaaagcggctctgaggtcagataaactaacggtggcggcgcgtggaaacggccattctatcaacggtcaagccatggcggaaggaggactcgttgtcgatatgagtaccacggcggagaatcatttcgaggttggttatttatccggcggtgatgccacggcgtttgttgatgtctccggaggggcattatgggaagatgtattgaaacggtgcgtttcggagtacggtttggctccgaggtcttggactgattatcttgggttaacggtgggaggtacgttgtcaaatgccggcgttagtggtcaagcgttccgttacggaccacagacgtcaaatgtaacggagttggacgtcgttacgggaaatggtgacgtcgttacttgctcggagattgagaattcagagctattcttctctgttttaggtggtcttggtcagtttggtatcatcaccagagctagggttttgctacagccagctcctgatatggtgaatacttaaaaccaacaatataaataacaatctcagttatatatatatatattttctctaatccaatcaaaaataagatatttggtccataatataaatgattgttgtgttaggtgagatggataagagtagtatacaccgagttcgatgagttcactcaagacgccgagtggctagtaagtcagaagaacgagtcatcgttcgattacgtggaaggattcgtgtttgtcaacggtgctgacccggttaacggatggccaacagttcccctccacccggaccacgagtttgacccgacccgactaccacaatcttgcgggtcggttctttattgcctcgaactcggtcttcactacagagactccgattccaactcaaccattgacaaggtaataataactttgagaaactttataacatttttcagaaattcaagaaccgttcatcttttatgatctaacggcggtggaagattctgatgttctagaaactttgtttgaccgaaattgaccttagattgaagtgtgaagttgacccgttttatttcactaactgttatacgacacgtagttcatataggaccgttttcagatttctcgacctccatgattacacaaacatacaattcaaaaaacagtaaaaagatgataataataataatatatggtttagttaaggaaatataatagggtgggaaagggaattatacagtctcttgtctgactgcataatatgaaactgacgagacattgtgtaatgtatcttcgattttggattgtctgacatgaaaaaaatatttatttgcttctctctaatgcccttgtcgtaaccacgttattacgaaaaggacatttgtcttcgttttctttttcttttctttttttttgttgcttttgttgtctttctcatgaaacacatattttaagatcactttgcctttttctactcaattatttagattaaaccaacacgtgtgacgtgtccattggtcgtgcggtatgggacgtaaggttgaaatcgtaattgtagcatgtaaacgtttctgtagtaaaacattgatgatatgatttcaaacggtcccggctaaaatctggccatcgttttatatggaatcatctatgtatgtaccgaaataccccctgactgatttttttccatttttttgtgtagagggtggagagattgatcggacggctaagatttaatgaaggattaagattcgaggtagatctgccgtacgttgactttttactacgagtcaaacggtcagaagaaatcgcgaaggagaacggtacgtgggaaacgcctcacccttggctcaacctcttcgtgtcgaagcgagacatcggagatttcaatcggacggtgttcaaagaacttgtcaagaacggagtcaatggtccaatgcttgtgtacccactcttgcgaagcaggtgaatattgctctctcttcctctcttttaactaggacccatctttttattttgggttagacaaggcacctatctaaaagactaaaagaagatcggtctagtttagttttatggcatgtgtttatcacgtgtgatgattagtcgtgcatgcttaaactaaaaaaaggctccacaagtcgcaagtcgtgtgatcaacaaattgtcgccaatgtggcacacgtgtctttcttcagtcccctcgtcattttttttacccgtacggggttttaagtacaataaaagttggaatttagtgtggtcgtttagattttgtaggcgagataaaaaaaagaatactaaactaatacgatgccgtattaggtattacggttgggtgggtgacggatatgtattgtaaccgtcgttaaggttagcgtcatatagggaaagagatgaaatttgtagggacccaatatgaactaacgttaaattttttattttacatttctaaaatagccttttgtaggttactgaagggtagtttcgtctttatatgttttactttatgtggaaaatgagatttgctggtacaaatagtagacgtaagaaatgaaaccaatcgtgagcaaagggccaccaaaatgtttattttttattctccgatttttttatggaaaatgtcttttgttccatttagatttagtgggtatttgttttataatatgaatgattaaataataatttggttggtttttatcaggtgggatgatcggacgtccgtggttataccggaagaaggagagatattctacattgtggcattgcttcggttcgtgccgccgtgtgcgaaagtctcttcggtagagaaaatggtagctcaaaaccaagagatcgttcattggtgtgtcaaaaacggaattgattacaaattgtatcttcctcattacaagtctcaagaggaatggattcgccattttggaaaccgatggtcgagatttgttgataggaaagctatgtttgatcccatggctatactttcaccgggtcaaaagattttcaataggtctctttga

SEQ ID NO: 13 - protein, 575 aa, Arabidopsis thaliana
MGLTSSLRFHRQNNKTFLGIFMILVLSCIPGRTNLCSNHSVSTPKELPSSNPSDIRSSLVSLDLEGYISFDDVHNVAKDFGNRYQLPPLAILHPRSVFDISSMMKHIVHLGSTSNLTVAARGHGHSLQGQALAHQGVVIKMESLRSPDIRIYKGKQPYVDVSGGEIWINILRETLKYGLSPKSWTDYLHLTVGGTLSNAGISGQAFKHGPQINNVYQLEIVTGKGEVVTCSEKRNSELFFSVLGGLGQFGIITRARISLEPAPHMVKWIRVLYSDFSAFSRDQEYLISKEKTFDYVEGFVIINRTDLLNNWRSSFSPNDSTQASRFKSDGKTLYCLEVVKYFNPEEASSMDQETGKLLSELNYIPSTLFSSEVPYIEFLDRVHIAERKLRAKGLWEVPHPWLNLLIPKSSIYQFATEVFNNILTSNNNGPILIYPVNQSKWKKHTSLITPNEDIFYLVAFLPSAVPNSSGKNDLEYLLKQNQRVMNFCAAANLNVKQYLPHYETQKEWKSHFGKRWETFAQRKQAYDPLAILAPGQRIFQKTTGKLSPIQLAKSKATGSPQRYHYASILPKPRTV

SEQ ID NO: 14 - DNA, 2236 bp, Arabidopsis thaliana
atgggattgacctcatccttacggttccatagacaaaacaacaagactttcctcggaatcttcatgatcttggttctaagctgtataccaggtagaaccaatctttgttccaatcattctgttagtaccccaaaagaattaccttcttcaaatccttcagatattcgttcctcattagtttcactagatttggagggttatataagcttcgacgatgtccacaatgtggccaaggactttggcaacagataccagttaccacctttggcaattctacatccaaggtcagtttttgatatttcatcgatgatgaagcatatagtacatctgggctccacctcaaatcttacagtagcagctagaggccatggtcactcgcttcaaggacaagctctagctcatcaaggtgttgtcatcaaaatggagtcacttcgaagtcctgatatcaggatttataaggggaagcaaccatatgttgatgtctcaggtggtgaaatatggataaacattctacgcgagactctaaaatacggtctttcaccaaagtcctggacagactaccttcatttgaccgttggaggtacactatctaatgctggaatcagcggtcaagcattcaagcatggaccccaaatcaacaacgtctaccagctagagattgttacaggtatttcattcatgctttatctctgcggtagtctcaaaaaaatatgcacctgtaaagaatatccatctcttcatgagcaaaaacactgacgactttaaataatttttgactataaaacaagagtgcataggcacaaatgtgaaatatgcaacacacaattgtaacttgcaccaagaaaaaagttataaaaacaaacaactgataagcaatatatttccaatatttaatcagggaaaggagaagtcgtaacctgttctgagaagcggaattctgaacttttcttcagtgttcttggcgggcttggacagtttggcataatcacccgggcacggatctctcttgaaccagcaccgcatatggtaaagttctatcttgaacaaagttcaaacaatatacgctatgattctaagaaccactttcctgacacagtcaaataacttttaataggttaaatggatcagggtactctactctgacttttctgcattttcaagggaccaagaatatctgatttcgaaggagaaaacttttgattacgttgaaggatttgtgataatcaatagaacagaccttctcaataattggcgatcgtcattcagtcccaacgattccacacaggcaagcagattcaagtcagatgggaaaactctttattgcctagaagtggtcaaatatttcaacccagaagaagctagctctatggatcaggtaagatgtgaaagcaatatataactagacttagtttccacagagagctccaaatcaaccgttggctactagcctactaacataatgaatggttgccgtgcaggaaactggcaagttactttcagagttaaattatattccatccactttgttttcatctgaagtgccatatatcgagtttctggatcgcgtgcatatcgcagagagaaaactaagagcaaagggtttatgggaggttccacatccctggctgaatctcctgattcctaagagcagcatataccaatttgctacagaagttttcaacaacattctcacaagcaacaacaacggtcctatccttatttatccagtcaatcaatccaagtaagtgagcaaaatgccaaaagcaaatgcgtccagtgattctgaaacataaattactaaccatatccaacattttgtggtttcaggtggaagaaacatacatctttgataactccaaatgaagatatattctatctcgtagcctttctcccctctgcagtgccaaattcctcagggaaaaacgatctagagtaccttttgaaacaaaaccaaagagttatgaacttctgcgcagcagcaaacctcaacgtgaagcagtatttgccccattatgaaactcaaaaagagtggaaatcacactttggcaaaagatgggaaacatttgcacagaggaaacaagcctacgaccctctagcgattctagcacctggccaaagaatattccaaaagacaacaggaaaattatctcccatccaactcgcaaagtcaaaggcaacaggaagtcctcaaaggtaccattacgcatcaatactgccgaaacctagaactgtataa

SEQ ID NO: 15 - DNA, 20 bp, Artificial Sequence
  Description of Artificial Sequence Synthetic primer
gaatggtggaattggtggtc

SEQ ID NO: 16 - DNA, 20 bp, Artificial Sequence
  Description of Artificial Sequence Synthetic primer
gcgagcatgtcaacatttca

SEQ ID NO: 17 - DNA, 22 bp, Artificial Sequence
  Description of Artificial Sequence Synthetic primer
tggttcacgtagtgggccatcg

SEQ ID NO: 18 - DNA, 20 bp, Artificial Sequence
  Description of Artificial Sequence Synthetic primer
tcaaaagcctcccaattgtc

SEQ ID NO: 19 - DNA, 20 bp, Artificial Sequence
  Description of Artificial Sequence Synthetic primer
ctcggctaaagacggagttg

SEQ ID NO: 20 - DNA, 22 bp, Artificial Sequence
  Description of Artificial Sequence Synthetic primer
tggttcacgtagtgggccatcg

SEQ ID NO: 21 - DNA, 26 bp, Artificial Sequence
  Description of Artificial Sequence Synthetic primer
ctctgccgcttctcacgacttcggta

SEQ ID NO: 22 - DNA, 26 bp, Artificial Sequence
  Description of Artificial Sequence Synthetic primer
cataaaccctggagcgaaacctagag

SEQ ID NO: 23 - DNA, 22 bp, Artificial Sequence
  Description of Artificial Sequence Synthetic primer
tggttcacgtagtgggccatcg

SEQ ID NO: 24 - DNA, 26 bp, Artificial Sequence
  Description of Artificial Sequence Synthetic primer
caaggtaaaactcacacgccataacc

SEQ ID NO: 25 - DNA, 26 bp, Artificial Sequence
  Description of Artificial Sequence Synthetic primer
cataaaccctggagcgaaacctagag

SEQ ID NO: 26 - DNA, 26 bp, Artificial Sequence
  Description of Artificial Sequence Synthetic primer
gagcgtcggtccccacacttctatac

SEQ ID NO: 27 - DNA, 29 bp, Artificial Sequence
  Description of Artificial Sequence Synthetic primer
ttgttgcagcaacgaccaaccgataatga

SEQ ID NO: 28 - DNA, 29 bp, Artificial Sequence
  Description of Artificial Sequence Synthetic primer
aatggtatattgtgatgacaggtgagatg

SEQ ID NO: 29 - DNA, 22 bp, Artificial Sequence
  Description of Artificial Sequence Synthetic primer
tggttcacgtagtgggccatcg

SEQ ID NO: 30 - DNA, 29 bp, Artificial Sequence
  Description of Artificial Sequence Synthetic primer
aatggtatattgtgatgacaggtgagatg

SEQ ID NO: 31 - DNA, 29 bp, Artificial Sequence
  Description of Artificial Sequence Synthetic primer
ttgttgcagcaacgaccaaccgataatga

SEQ ID NO: 32 - DNA, 23 bp, Artificial Sequence
  Description of Artificial Sequence Synthetic primer
atattgaccatcatactcattgc

SEQ ID NO: 33 - DNA, 21 bp, Artificial Sequence
  Description of Artificial Sequence Synthetic primer
accctgtccaagaatgcttca

SEQ ID NO: 34 - DNA, 21 bp, Artificial Sequence
  Description of Artificial Sequence Synthetic primer
tgtggattcccctgctccata

SEQ ID NO: 35 - DNA, 22 bp, Artificial Sequence
  Description of Artificial Sequence Synthetic primer
tggttcacgtagtgggccatcg

SEQ ID NO: 36 - DNA, 20 bp, Artificial Sequence
  Description of Artificial Sequence Synthetic primer
ttagccgtccgatcaatctc

SEQ ID NO: 37 - DNA, 20 bp, Artificial Sequence
  Description of Artificial Sequence Synthetic primer
cggaaaatctacggatggtg

SEQ ID NO: 38 - DNA, 23 bp, Artificial Sequence
  Description of Artificial Sequence Synthetic primer
atattgaccatcatactcattgc

SEQ ID NO: 39 - DNA, 27 bp, Artificial Sequence
  Description of Artificial Sequence Synthetic primer
gctagtaagtcagaagaacgagtcatc

SEQ ID NO: 40 - DNA, 20 bp, Artificial Sequence
  Description of Artificial Sequence Synthetic primer
ttagccgtccgatcaatctc

SEQ ID NO: 41 - DNA, 34 bp, Artificial Sequence
  Description of Artificial Sequence Synthetic primer
gccttttcagaaatggataaatagccttgcttcc

SEQ ID NO: 42 - DNA, 20 bp, Artificial Sequence
  Description of Artificial Sequence Synthetic primer
gaatggtggaattggtggtc

SEQ ID NO: 43 - DNA, 20 bp, Artificial Sequence
  Description of Artificial Sequence Synthetic primer
agtcccgaagctgatttttg

SEQ ID NO: 44 - DNA, 20 bp, Artificial Sequence
  Description of Artificial Sequence Synthetic primer
ctcggctaaagacggagttg

SEQ ID NO: 45 - DNA, 26 bp, Artificial Sequence
  Description of Artificial Sequence Synthetic primer
aataggtggttgtaaacgtagacgca

SEQ ID NO: 46 - DNA, 26 bp, Artificial Sequence
  Description of Artificial Sequence Synthetic primer
ctctgccgcttctcacgacttcggta

SEQ ID NO: 47 - DNA, 26 bp, Artificial Sequence
  Description of Artificial Sequence Synthetic primer
cataaaccctggagcgaaacctagag

SEQ ID NO: 48 - DNA, 26 bp, Artificial Sequence
  Description of Artificial Sequence Synthetic primer
ctctgccgcttctcacgacttcggta

SEQ ID NO: 49 - DNA, 26 bp, Artificial Sequence
  Description of Artificial Sequence Synthetic primer
cataaaccctggagcgaaacctagag

SEQ ID NO: 50 - DNA, 20 bp, Artificial Sequence
  Description of Artificial Sequence Synthetic primer
gcacgaatctctctcgaacc

SEQ ID NO: 51 - DNA, 19 bp, Artificial Sequence
  Description of Artificial Sequence Synthetic primer
cgctgacgaagaagacgac

SEQ ID NO: 52 - DNA, 20 bp, Artificial Sequence
  Description of Artificial Sequence Synthetic primer
gcacgaatctctctcgaacc

SEQ ID NO: 53 - DNA, 20 bp, Artificial Sequence
  Description of Artificial Sequence Synthetic primer
aaattcttggaccggagctt

SEQ ID NO: 54 - DNA, 21 bp, Artificial Sequence
  Description of Artificial Sequence Synthetic primer
tgtggattcccctgctccata

SEQ ID NO: 55 - DNA, 21 bp, Artificial Sequence
  Description of Artificial Sequence Synthetic primer
accctgtccaagaatgcttca

SEQ ID NO: 56 - DNA, 20 bp, Artificial Sequence
  Description of Artificial Sequence Synthetic primer
ttagccgtccgatcaatctc

SEQ ID NO: 57 - DNA, 20 bp, Artificial Sequence
  Description of Artificial Sequence Synthetic primer
cggaaaatctacggatggtg

SEQ ID NO: 58 - DNA, 20 bp, Artificial Sequence
  Description of Artificial Sequence Synthetic primer
ttagccgtccgatcaatctc

SEQ ID NO: 59 - DNA, 20 bp, Artificial Sequence
  Description of Artificial Sequence Synthetic primer
cggaaaatctacggatggtg

SEQ ID NO: 60 - DNA, 20 bp, Artificial Sequence
  Description of Artificial Sequence Synthetic primer
tacaacgagcttcgtgttgc

SEQ ID NO: 61 - DNA, 20 bp, Artificial Sequence
  Description of Artificial Sequence Synthetic primer
gattgatcctccgatccaga